(19) United States
(12) Patent Application Publication
(10) Pub. No.: US 2025/0034597 A1
(43) Pub. Date: Jan. 30, 2025

(54) RNA-Guided Targeting of Genetic and Epigenomic Regulatory Proteins to Specific Genomic Loci

(71) Applicant: The General Hospital Corporation, Boston, MA (US)

(72) Inventors: J. Keith Joung, Winchester, MA (US); Morgan Maeder, Brookline, MA (US)

(21) Appl. No.: 18/764,634

(22) Filed: Jul. 5, 2024

Related U.S. Application Data
(63) Continuation of application No. 17/450,135, filed on Oct. 6, 2021, now Pat. No. 12,065,668.
Application No. 17/450,135 is a division of application No. 16/535,199, filed on Aug. 8, 2019, now Pat. No. 11,168,338.
Application No. 16/535,199 is a continuation of application No. 14/775,869, filed on Sep. 14, 2015, now Pat. No. 10,378,027.
(60) Provisional application No. 61/799,647, filed on Mar. 15, 2013.
(60) Provisional application No. 61/838,178, filed on Jun. 21, 2013.
(60) Provisional application No. 61/838,148, filed on Jun. 21, 2013.
(60) Provisional application No. 61/921,007, filed on Dec. 26, 2013.

(51) Int. Cl. C12N 15/90; C07K 14/005; C07K 14/195; C12N 9/02; C12N 9/10; C12N 9/16; C12N 9/22; C12N 9/96; C12N 15/01; C12N 15/10; C12N 15/11; C12N 15/63; C12N 15/85

(52) U.S. Cl. CPC C12N 15/907; C07K 14/005; C07K 14/195; C12N 9/0071; C12N 9/1007; C12N 9/16; C12N 9/22; C12N 9/96; C12N 15/01; C12N 15/102; C12N 15/1031; C12N 15/11; C12N 15/63; C12N 15/85; C12Y 301/21004; C07K 2319/00; C07K 2319/01; C07K 2319/80; C12N 2310/20; C12N 2710/00033; C12N 2770/00033; C12N 2800/80; C12Y 114/11; C12Y 201/01; C12Y 301/00

(57) ABSTRACT

Methods and constructs for RNA-guided targeting of heterologous functional domains such as transcriptional activators to specific genomic loci.

### CLAIM OF PRIORITY

This application is a continuation of U.S. patent application Ser. No. 17/450,135, filed Oct. 6, 2021, which is a divisional of U.S. patent application Ser. No. 16/535,199, filed Aug. 8, 2019, now U.S. Pat. No. 11,168,338, which is a continuation of U.S. patent application Ser. No. 14/775,869, filed Sep. 14, 2015, now U.S. Pat. No. 10,378,027, which is a U.S. National Phase Application under 35 U.S.C. § 371 of International Patent Application No. PCT/US2014/027335, filed on Mar. 14, 2014, which claims the benefit of U.S. Patent Application Ser. Nos. 61/799,647, filed on Mar. 15, 2013; 61/838, 178, filed on Jun. 21, 2013; 61/838, 148, filed on Jun. 21, 2013; and 61/921,007, filed on Dec. 26, 2013. The entire contents of the foregoing are hereby incorporated by reference.

### FEDERALLY SPONSORED RESEARCH OR DEVELOPMENT

This invention was made with Government support under Grant No. DP1GM105378 awarded by the National Institutes of Health and W911NF-Nov. 2, 0056 awarded by the Defense Advanced Research Projects Agency (DARPA) of the Department of Defense. The Government has certain rights in the invention.

### SEQUENCE LISTING

This application contains a Sequence Listing that has been submitted electronically as an XML file named “40174-0006005_SL_ST26.XML.” The XML file, created on Jul. 3, 2024, is 129,216 bytes in size. The material in the XML file is hereby incorporated by reference in its entirety.

### TECHNICAL FIELD

This invention relates to methods and constructs for RNA-guided targeting of genetic and epigenomic regulatory proteins, e.g., transcriptional activators, histone modification enzymes, DNA methylation modifiers, to specific genomic loci.

### BACKGROUND

Clustered Regulatory Interspaced Short Palindromic Repeats (CRISPR), and CRISPR-associated (cas) genes, referred to as CRISPR/Cas systems, are used by various bacteria and archaea to mediate defense against viruses and other foreign nucleic acid. These systems use small RNAs to detect and silence foreign nucleic acids in a sequence-specific manner.

Three types of CRISPR/Cas systems have been described (Makarova et al., Nat. Rev. Microbiol. 9, 467 (2011); Makarova et al., Biol. Direct 1, 7 (2006); Makarova et al., Biol. Direct 6, 38 (2011)). Recent work has shown that Type II CRISPR/Cas systems can be engineered to direct targeted double-stranded DNA breaks in vitro to specific sequences by using a single “guide RNA” with complementarity to the DNA target site and a Cas9 nuclease (Jinek et al., Science 2012; 337:816-821). This targetable Cas9-based system also works in cultured human cells (Mali et al., Science. 2013 Feb. 15; 339(6121):823-6; Cong et al., Science. 2013 Feb. 15; 339(6121):819-23) and in vivo in zebrafish (Hwang and Fu et al., Nat Biotechnol. 2013 March; 31(3):227-9) for inducing targeted alterations into endogenous genes.

### SUMMARY

At least in part, the present invention is based on the development of a fusion protein including a heterologous functional domain (e.g., a transcriptional activation domain) fused to a Cas9 nuclease that has had its nuclease activity inactivated by mutations (also known as “dCas9”). While published studies have used guide RNAs to target catalytically active and inactive Cas9 nuclease proteins to specific genomic loci, no work has yet adapted the use of this system to recruit additional effector domains. This work also provides the first demonstration of an RNA-guided process that results in an increase (rather than a decrease) in the level of expression of a target gene.

In addition, the present disclosure provides the first demonstration that multiplex gRNAs can be used to bring multiple dCas9-VP64 fusions to a single promoter, thereby resulting in synergistic activation of transcription.

Thus, in a first aspect, the invention provides fusion proteins comprising a catalytically inactive CRISPR associated 9 (dCas9) protein linked to a heterologous functional domain (HFD) that modifies gene expression, histones, or DNA, e.g., transcriptional activation domain, transcriptional repressors (e.g., silencers such as Heterochromatin Protein 1 (HP1), e.g., HP1α or HP1β, or a transcriptional repression domain, e.g., Krueppel-associated box (KRAB) domain, ERF repressor domain (ERD), or mSin3A interaction domain (SID)), enzymes that modify the methylation state of DNA (e.g., DNA methyltransferase (DNMT) or Ten-Eleven Translocation (TET) proteins, e.g., TET1, also known as Tet Methylcytosine Dioxygenase 1), or enzymes that modify histone subunit (e.g., histone acetyltransferases (HAT), histone deacetylases (HDAC), or histone demethylases). In some embodiments, the heterologous functional domain is a transcriptional activation domain, e.g., a transcriptional activation domain from VP64 or NF-κB p65; an enzyme that catalyzes DNA demethylation, e.g., a TET; or histone modification (e.g., LSD1, histone methyltransferase, HDACs, or HATs) or a transcription silencing domain, e.g., from Heterochromatin Protein 1 (HP1), e.g., HP1α or HP1β; or a biological tether, e.g., CRISPR/Cas Subtype Ypest protein 4 (Csy4), MS2,or lambda N protein.

In some embodiments, the catalytically inactive Cas9 protein is from S. pyogenes.

In some embodiments, the catalytically inactive Cas9 protein comprises mutations at comprises mutations at D10, E762, H983, or D986; and at H840 or N863, e.g., at D10 and H840, e.g., D10A or D1ON and H840A or H840N or H840Y.

In some embodiments, the heterologous functional domain is linked to the N terminus or C terminus of the catalytically inactive Cas9 protein, with an optional intervening linker, wherein the linker does not interfere with activity of the fusion protein.

In some embodiments, the fusion protein includes one or both of a nuclear localization sequence and one or more epitope tags, e.g., c-myc, 6His, or FLAG tags, on the N-terminus, C-terminus, or in between the catalytically inactive CRISPR associated 9 (Cas9) protein and the heterologous functional domain, optionally with one or more intervening linkers.

In further aspect, the invention provides nucleic acids encoding the fusion proteins described herein, as well as expression vectors including the nucleic acids, and host cells expressing the fusion proteins.

In an additional aspect, the invention provides methods for increasing expression of a target gene in a cell. The methods include expressing a Cas9-HFD fusion protein as described herein in the cell, e.g., by contacting the cell with an expression vector including a sequence encoding the fusion protein, and also expressing in the cell one or more guide RNAs with complementarity directed to the target gene, e.g., by contacting the cell with one or more expression vectors comprising nucleic acid sequences encoding one or more guide RNAs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a schematic illustration showing a single guide RNA (sgRNA) recruiting Cas9 nuclease to a specific DNA sequence and thereby introducing targeted alterations. The sequence of the guide RNA shown is

(SEQ ID NO: 9)
GGAGCGAGCGGAGCGGUACAGUUUUAGAGCUAGAAAUAGCAAGUUAAAA
UAAGGCUAGUCCG

FIG. 1B is a schematic illustration showing a longer version of the sgRNA used to recruit Cas9 nuclease to a specific DNA sequence and to thereby introduce targeted alterations. The sequence of the guide RNA shown is

(SEQ ID NO: 10)
GGAGCGAGCGGAGCGGUACAGUUUUAGAGCUAGAAAUAGCAAGUUAAAA
UAAGGCUAGUCCGUUAUCAACUUGAAAAAGUGGCACCGAGUCGGUGCUU
UU.

FIG. 1C is a schematic illustration showing a Cas9 protein containing D10A and H840A mutations to render the nuclease portion of the protein catalytically inactive, fused to a transcriptional activation domain and recruited to a specific DNA sequence by a sgRNA. The sequence of the guide RNA shown is

(SEQ ID NO: 10)
GGAGCGAGCGGAGCGGUACAGUUUUAGAGCUAGAAAUAGCAAGUUAAAA
UAAGGCUAGUCCGUUAUCAACUUGAAAAAGUGGCACCGAGUCGGUGCUU
UU.

FIG. 1D is a schematic depicting recruitment of dCas9-VP64 fusion protein to a specific genomic target sequence by a chimeric sgRNA.

FIG. 1E is a diagram illustrating the positions and orientations of 16 sgRNAs targeted to the endogenous human VEGFA gene promoter. Small horizontal arrows represent the first 20 nts of the gRNA complementary to the genomic DNA sequence with the arrow pointing 5′ to 3′. Grey bars indicate DNasel hypersensitive sites previously defined in human 293 cells (Liu et al., J Biol Chem. 2001 Apr. 6; 276(14):11323-34), numbered relative to the transcription start site (right-angle arrow).

FIG. 2A is a bar graph showing activation of VEGFA protein expression in 293 cells by various sgRNAs, each expressed with (grey bars) or without (black bars) dCas9-VP64. Fold-activation of VEGFA was calculated relative to the off-target sgRNA control as described in Methods. Each experiment was performed in triplicate and error bars represent standard errors of the mean. Asterisks indicate samples that are significantly elevated above the off-target control as determined by a paired, one-sided t-test (p<0.05).

FIG. 2B is a bar graph showing multiplex sgRNA expression induces synergistic activation of VEGFA protein expression by dCas9-VP64 protein. Fold-activation of VEGFA protein in 293 cells in which the indicated combinations of sgRNAs were co-expressed with dCas9-VP64 is shown. Note that in all of these experiments the amount of each individual sgRNA expression plasmid used for transfection was the same. Fold-activation values were calculated as described in 2A and shown as grey bars. The calculated sum of mean fold-activation values induced by individual sgRNAs is shown for each combination as black bars. Asterisks indicate all combinations that were found to be significantly greater than the expected sum as determined by an analysis of variance (ANOVA) (p<0.05).

FIG. 3A is a diagram illustrating the positions and orientations of six sgRNAs targeted to the endogenous human NTI3 gene promoter. Horizontal arrows represent the first 20 nts of the sgRNA complementary to the genomic DNA sequence with the arrow pointing 5′ to 3′. Grey line indicates region of potential open chromatin identified from the ENCODE DNasel hypersensitivity track on the UCSC genome browser with the thicker part of the bar indicating the first transcribed exon. Numbering shown is relative to the transcription start site (+1, right-angle arrow).

FIG. 3B is a bar graph showing activation of NTI 3 gene expression by sgRNA-guided dCas9-VP64 in 293 cells. Relative expression of NTF3 mRNA, detected by quantitative RT-PCR and normalized to a GAPDH control (deltaCt×10⁴), is shown for 293 cells co-transfected with the indicated amounts of dCas9-VP64 and NTI 3-targeted sgRNA expression plasmids. All experiments were performed in triplicate with error bars representing standard errors of the mean. Asterisks indicate samples that are significantly greater than the off-target gRNA control as determined by a paired, one-sided T-test (P<0.05).

FIG. 3C is a bar graph showing multiplex gRNA expression induces synergistic activation of NTF3 mRNA expression by dCas9-VP64 protein. Relative expression of NTF3 mRNA, detected by quantitative RT-PCR and normalized to a GAPDH control (deltaCt×10⁴), is shown for 293 cells co-transfected with dCas9-VP64 and the indicated combinations of NTF3-targeted gRNA expression plasmids. Note that in all of these experiments the amount of each individual gRNA expression plasmid used for transfection was the same. All experiments were performed in triplicate with error bars representing standard errors of the mean. The calculated sum of mean fold-activation values induced by individual gRNAs is shown for each combination.

FIG. 4 is an exemplary sequence of an sgRNA expression vector.

FIG. 5 is an exemplary sequence of CMV-T7-Cas9 D10A/H840A-3×FLAG-VP64 expression vector.

FIG. 6 is an exemplary sequence of CMV-T7-Cas9 recoded D10A/H840A-3×FLAG-VP64 expression vector.

FIG. 7 is an exemplary sequence of a Cas9-HFD, i.e., a Cas9-activator. An optional 3×FLAG sequence is underlined; the nuclear localization signal PKKKRKVS (SEQ ID NO: 11) is in lower case; two linkers are in bold; and the VP64 transcriptional activator sequence,

DALDDFDLDMLGSDALDDFDLDMLGSDALDDFDLDMLGSDALDDFDLDML (SEQ ID NO: 12), is boxed.

FIGS. 8A-8B are exemplary sequences of (8A) dCas9-NLS-3×FLAG-HP1alpha and (8B) dCas9-NLS-3×FLAG-HP1β eta. Box=nuclear localization signal; underline=triple flag tag; double underline=HP 1 alpha hinge and chromoshadow domains.

FIG. 9 is an exemplary sequence of dCas9-TET1.

FIG. 10 is a bar graph showing results obtained with various dCas9-VP64 fusion constructs. Of those tested, the optimized dCas9-VP64 architecture included an N-terminal NLS (NFN) and an additional NLS (N) or FLAG tag/NLS (NF) placed between dCas9 and VP64. Expression of the VEGFA gene in human HEK293 cells was activated by transcriptional activation mediated by RNA-guided dCas9-VP64 fusions. Expression plasmids encoding variants of dCas9-VP64 were co-transfected with a plasmid that expressed three gRNAs that targeted sites in a region upstream of the VEGFA start codon (in this experiment, the gRNAs were expressed from a single gRNA and processed out by the Csy4 endoribonuclease). VEGFA protein expression is measured by ELISA, and the mean of two replicates is shown with error bars indicating standard errors of the mean.

FIGS. 11A-B are bar graphs showing the activities of dCas9-VP64 activators bearing alternative substitution mutations to catalytically inactivate Cas9 function. (11A) Plasmids expressing dCas9-VP64 proteins bearing various Cas9 inactivating substitutions to residues D10 and H840 were each co-transfected into HEK293 cells with either a single gRNA or three distinctly targeted gRNAs targeting the VEGFA upstream region (blue and red bars, respectively). (11B) Plasmids expressing these dCas9-VP64 variants were also transfected into a HEK293 cell-line that stably expresses a single VEGFA-targeted gRNA. VEGFA protein levels were determined by ELISA with mean of two replicates and standard errors of the mean (error bars) shown.

### DETAILED DESCRIPTION

Described herein are fusion proteins of a heterologous functional domain (e.g., a transcriptional activation domain) fused to a catalytically inactivated version of the Cas9 protein for the purpose of enabling RNA-guided targeting of these functional domains to specific genomic locations in cells and living organisms.

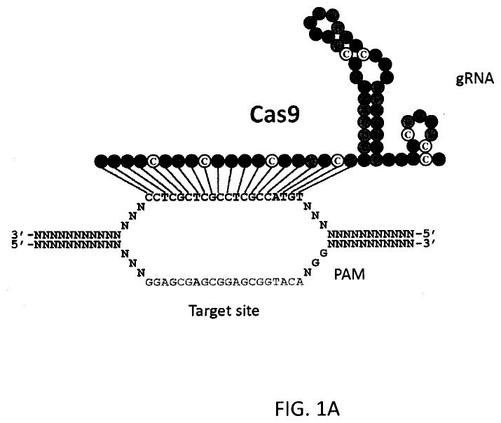

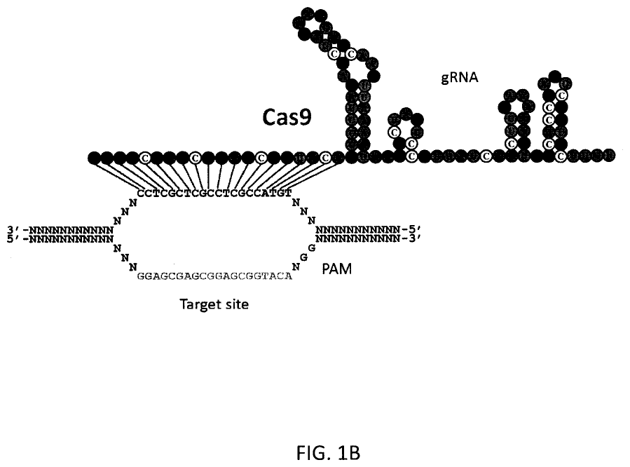

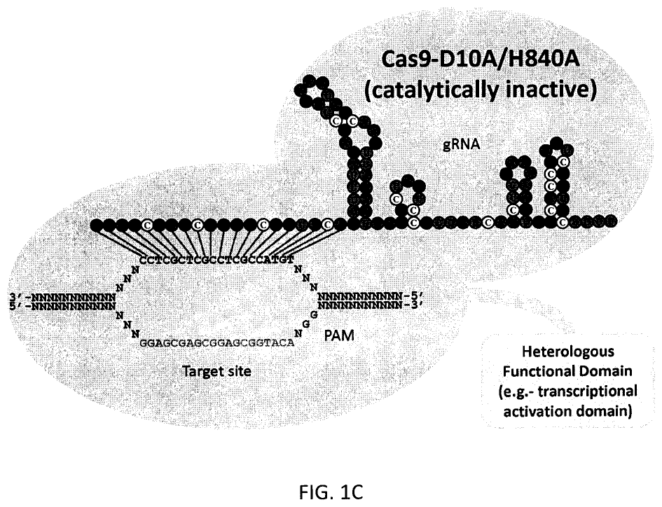

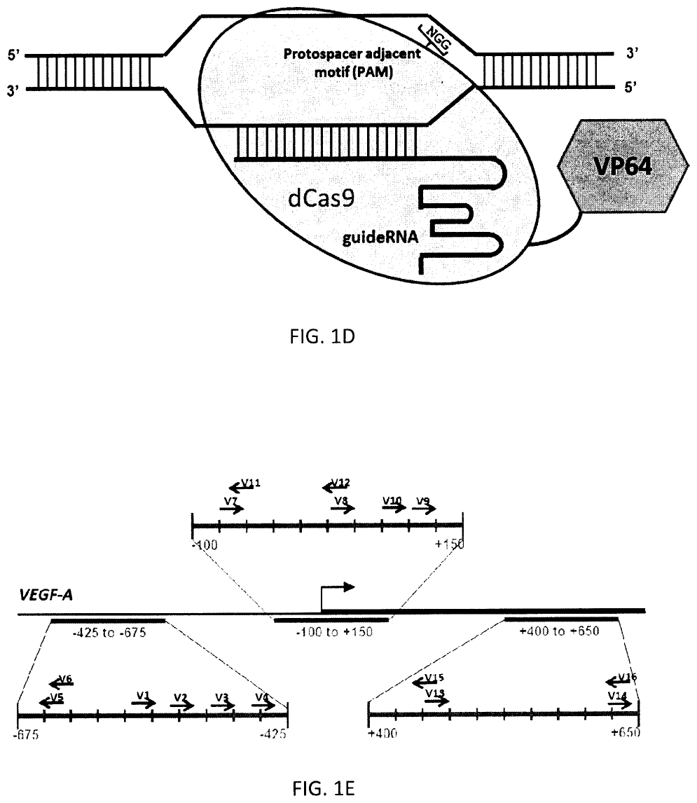

The CRISPR/Cas system has evolved in bacteria as a defense mechanism to protect against invading plasmids and viruses. Short protospacers, derived from foreign nucleic acid, are incorporated into CRISPR loci and subsequently transcribed and processed into short CRISPR RNAs (crRNAs). These crRNAs, complexed with a second tracrRNA, then use their sequence complementarity to the invading nucleic acid to guide Cas9-mediated cleavage, and consequent destruction of the foreign nucleic acid. In 2012, Doudna and colleagues demonstrated that a single guide RNA (sgRNA) composed of a fusion of a crRNA with tracrRNA can mediate recruitment of Cas9 nuclease to specific DNA sequences in vitro (FIG. 1C; Jinek et al., Science 2012).

More recently, a longer version of the sgRNA has been used to introduce targeted alterations in human cells and zebrafish (FIG. 1B; Mali et al. Science 2013, Hwang and Fu et al., Nat Biotechnol. 2013 March; 31(3):227-9). Qi et al. demonstrated that gRNA-mediated recruitment of a catalytically inactive mutant form of Cas9 (referred to as dCas9) could lead to repression of specific endogenous genes in E. coli as well as of an EGFP reporter gene in human cells (Qi et al., Cell 152, 1173-1183 (2013)). Although this study demonstrated the potential to adapt RNA-guided Cas9 technology for regulation of gene expression, it did not test or demonstrate whether heterologous functional domains (e.g.—transcriptional activation domains) could be fused to dCas9 without disrupting its ability to be recruited to specific genomic sites by programmable sgRNAs or dual gRNAs (dgRNAs—i.e.—a customized crRNA and a tracrRNA).

As described herein, in addition to guiding Cas9-mediated nuclease activity, it is possible to use CRISPR-derived RNAs to target heterologous functional domains fused to Cas9 (Cas9-HFD) to specific sites in the genome (FIG. 1C). For example, as described herein, it is possible to use single guide RNAs (sgRNAs) to target Cas9-HFD, e.g., Cas9-transcriptional activators (hereafter referred to as Cas9-activators) to the promoters of specific genes and thereby increase expression of the target gene. Thus Cas9-HFD can be localized to sites in the genome, with target specificity defined by sequence complementarity of the guide RNA. The target sequence also includes a PAM sequence (a 2-5 nucleotide sequence specified by the Cas9 protein which is adjacent to the sequence specified by the RNA).

The Cas9-HFD are created by fusing a heterologous functional domain (e.g., a transcriptional activation domain, e.g., from VP64 or NF-κB p65), to the N-terminus or C-terminus of a catalytically inactive Cas9 protein.

### Cas9

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11):1116-21 and Fonfara et al., “Phylogeny of Cas9 determines functional exchangeability of dual-RNA and Cas9 among orthologous type II CRISPR-Cas systems.” Nucleic Acids Res. 2013 Nov. 22. [Epub ahead of print] doi: 10.1093/nar/gkt1074.

Cas9 molecules of a variety of species can be used in the methods and compositions described herein. While the S. pyogenes and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them. Such species include those set forth in the following table, which was created based on supplementary figure 1 of Chylinski et al., 2013.

Alternative Cas9 proteins

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

The constructs and methods described herein can include the use of any of those Cas9 proteins, and their corresponding guide RNAs or other guide RNAs that are compatible. The Cas9 from Streptococcus thermophilus LMD-9 CRISPR1 system has been shown to function in human cells in Cong et al (Science 339, 819 (2013)). Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency.

In some embodiments, the present system utilizes the Cas9 protein from S. pyogenes, either as encoded in bacteria or codon-optimized for expression in mammalian cells, containing mutations at D10, E762, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)) or they could be other residues, e.g., glutamine, asparagine, tyrosine, serine, or aspartate, e.g., E762Q, H983N, H983Y, D986N, N863D, N863S, or N863H (FIG. 1C). The sequence of the catalytically inactive S. pyogenes Cas9 that can be used in the methods and compositions described herein is as follows; the exemplary mutations of D10A and H840A are in bold and underlined.

```
(SEQ ID NO: 13)
                10 20 30 40 50 60
MDKKYSIGLA IGTNSVGWAV ITDEYKVPSK KFKVLGNTDR HSIKKNLIGA LLFDSGETAE
                70 80 90 100 110 120
ATRLKRTARR RYTRRKNRIC YLQEIFSNEM AKVDDSFFHR LEESFLVEED KKHERHPIFG
              130 140 150 160 170 180
NIVDEVAYHE KYPTIYHLRK KLVDSTDKAD LRLIYLALAH MIKFRGHFLI EGDLNPDNSD
              190 200 210 220 230 240
VDKLFIQLVQ TYNQLFEENP INASGVDAKA ILSARLSKSR RLENLIAQLP GEKKNGLFGN
              250 260 270 280 290 300
LIALSLGLTP NFKSNFDLAE DAKLQLSKDT YDDDLDNLLA QIGDQYADLF LAAKNLSDAI
              310 320 330 340 350 360
LLSDILRVNT EITKAPLSAS MIKRYDEHHQ DLTLLKALVR QQLPEKYKEI FFDQSKNGYA
              370 380 390 400 410 420
GYIDGGASQE EFYKFIKPIL EKMDGTEELL VKLNREDLLR KQRTFDNGSI PHQIHLGELH
              430 440 450 460 470 480
AILRRQEDFY PFLKDNREKI EKILTFRIPY YVGPLARGNS RFAWMTRKSE ETITPWNFEE
              490 500 510 520 530 540
VVDKGASAQS FIERMTNFDK NLPNEKVLPK HSLLYEYFTV YNELTKVKYV TEGMRKPAFL
              550 560 570 580 590 600
SGEQKKAIVD LLFKTNRKVT VKQLKEDYFK KIECFDSVEI SGVEDRFNAS LGTYHDLLKI
              610 620 630 640 650 660
IKDKDFLDNE ENEDILEDIV LTLTLFEDRE MIEERLKTYA HLFDDKVMKQ LKRRRYTGWG
              670 680 690 700 710 720
RLSRKLINGI RDKQSGKTIL DFLKSDGFAN RNFMQLIHDD SLTFKEDIQK AQVSGQGDSL
              730 740 750 760 770 780
HEHIANLAGS PAIKKGILQT VKVVDELVKV MGRHKPENIV IEMARENQTT QKGQKNSRER
              790 800 810 820 830 840
MKRIEEGIKE LGSQILKEHP VENTQLQNEK LYLYYLQNGR DMYVDQELDI NRLSDYDVDA
              850 860 870 880 890 900
IVPQSFLKDD SIDNKVLTRS DKNRGKSDNV PSEEVVKKMK NYWRQLLNAK LITQRKFDNL
              910 920 930 940 950 960
TKAERGGLSE LDKAGFIKRQ LVETRQITKH VAQILDSRMN TKYDENDKLI REVKVITLKS
              970 980 990 1000 1010 1020
KLVSDFRKDF QFYKVREINN YHHAHDAYLN AVVGTALIKK YPKLESEFVY GDYKVYDVRK
            1030 1040 1050 1060 1070 1080
MIAKSEQEIG KATAKYFFYS NIMNFFKTEI TLANGEIRKR PLIETNGETG EIVWDKGRDF
            1090 1100 1110 1120 1130 1140
ATVRKVLSMP QVNIVKKTEV QTGGFSKESI LPKRNSDKLI ARKKDWDPKK YGGFDSPTVA
            1150 1160 1170 1180 1190 1200
YSVLVVAKVE KGKSKKLKSV KELLGITIME RSSFEKNPID FLEAKGYKEV KKDLIIKLPK
            1210 1220 1230 1240 1250 1260
YSLFELENGR KRMLASAGEL QKGNELALPS KYVNFLYLAS HYEKLKGSPE DNEQKQLFVE
            1270 1280 1290 1300 1310 1320
QHKHYLDEII EQISEFSKRV ILADANLDKV LSAYNKHRDK PIREQAENII HLFTLTNLGA
            1330 1340 1350 1360
PAAFKYFDTT IDRKRYTSTK EVLDATLIHQ SITGLYETRI DLSQLGGD
```

In some embodiments, the Cas9 nuclease used herein is at least about 50% identical to the sequence of S. pyogenes Cas9, i.e., at least 50% identical to SEQ ID NO:13. In some embodiments, the nucleotide sequences are about 50%, 55%, 60%, 65%, 70%, 75%, 80%, 85%, 90%, 95%, 99% or 100% identical to SEQ ID NO:13.

In some embodiments, the catalytically inactive Cas9 used herein is at least about 50% identical to the sequence of the catalytically inactive S. pyogenes Cas9, i.e., at least 50%, 55%, 60%, 65%, 70%, 75%, 80%, 85%, 90%, 95%, 99% or 100% identical to SEQ ID NO: 13, wherein the mutations at D10 and H840, e.g., D10A/D1ON and H840A/H840N/H840Y are maintained.

In some embodiments, any differences from SEQ ID NO: 13 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11):1116-21 and Fonfara et al., Nucl. Acids Res. (2014) 42 (4): 2577-2590. [Epub ahead of print 2013 Nov. 22] doi:10.1093/nar/gkt1074, and wherein the mutations at D10 and H840, e.g., D10A/DION and H840A/H840N/H840Y are maintained.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For purposes of the present application, the percent identity between two amino acid sequences is determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### Heterologous Functional Domains

The transcriptional activation domains can be fused on the N or C terminus of the Cas9. In addition, although the present description exemplifies transcriptional activation domains, other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (IncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET) 1-3 family, enzymes that converts 5-methylcytosine (5-mC) to 5-hydroxymethylcytosine (5-hmC) in DNA.

Sequences for human TET1-3 are known in the art and are shown in the following table:

GenBank Accession Nos.

| Gene | Amino Acid | Nucleic Acid |
|---|---|---|
| TET1 | NP_085128.2 | NM_030625.2 |
| TET2* | NP_001120680.1 (var 1) | NM_001127208.2 |
| | NP_060098.3 (var2) | NM_017628.4 |
| TET3 | NP_659430.1 | NM_144993.1 |

*Variant (1) represents the longer transcript and encodes the longer isoform (a). Variant (2) differs in the 5′ UTR and in the 3′ UTR and coding sequence compared to variant 1. The resulting isoform (b) is shorter and has a distinct C-terminus compared to isoform a.

In some embodiments, all or part of the full-length sequence of the catalytic domain can be included, e.g., a catalytic module comprising the cysteine-rich extension and the 20GFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 fused to MS2 coat protein, endoribonuclease Csy4, or lambda N can be used to recruit a long non-coding RNA (IncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 binding site using the methods and compositions described herein. In some embodiments, the Csy4 is catalytically inactive.

In some embodiments, the fusion proteins include a linker between the dCas9 and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:14) or GGGGS (SEQ ID NO: 15), e.g., two, three, four, or more repeats of the GGGS (SEQ ID NO:14) or GGGGS (SEQ ID NO:15) unit. Other linker sequences can also be used.

### Methods of Use

The described Cas9-HFD system is a useful and versatile tool for modifying the expression of endogenous genes. Current methods for achieving this require the generation of novel engineered DNA-binding proteins (such as engineered zinc finger or transcription activator-like effector DNA binding domains) for each site to be targeted. Because these methods demand expression of a large protein specifically engineered to bind each target site, they are limited in their capacity for multiplexing. Cas9-HFD, however, require expression of only a single Cas9-HFD protein, which can be targeted to multiple sites in the genome by expression of multiple short gRNAs. This system could therefore easily be used to simultaneously induce expression of a large number of genes or to recruit multiple Cas9-HFDs to a single gene, promoter, or enhancer. This capability will have broad utility, e.g., for basic biological research, where it can be used to study gene function and to manipulate the expression of multiple genes in a single pathway, and in synthetic biology, where it will enable researchers to create circuits in cell that are responsive to multiple input signals. The relative ease with which this technology can be implemented and adapted to multiplexing will make it a broadly useful technology with many wide-ranging applications.

The methods described herein include contacting cells with a nucleic acid encoding the Cas9-HFD described herein, and nucleic acids encoding one or more guide RNAs directed to a selected gene, to thereby modulate expression of that gene.

Guide RNAs (gRNAs)

Guide RNAs generally speaking come in two different systems: System 1, which uses separate crRNA and tracrRNAs that function together to guide cleavage by Cas9, and System 2, which uses a chimeric crRNA-tracrRNA hybrid that combines the two separate guide RNAs in a single system (referred to as a single guide RNA or sgRNA, see also Jinek et al., Science 2012; 337:816-821). The tracrRNA can be variably truncated and a range of lengths has been shown to function in both the separate system (system 1) and the chimeric gRNA system (system 2). For example, in some embodiments, tracrRNA may be truncated from its 3′ end by at least 1, 2, 3, 4, 5, 6, 7, 8, 9, 10, 15, 20, 25, 30, 35 or 40 nts. In some embodiments, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. See, e.g., Jinek et al., Science 2012; 337:816-821; Mali et al., Science. 2013 Feb. 15; 339 (6121):823-6; Cong et al., Science. 2013 Feb. 15; 339(6121):819-23; and Hwang and Fu et al., Nat Biotechnol. 2013 March; 31 (3): 227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene.

Cas9 nuclease can be guided to specific 17-20 nt genomic targets bearing an additional proximal protospacer adjacent motif (PAM), e.g., of sequence NGG, using a guide RNA, e.g., a single gRNA or a tracrRNA/crRNA, bearing 17-20 nts at its 5′ end that are complementary to the complementary strand of the genomic DNA target site. Thus, the present methods can include the use of a single guide RNA comprising a crRNA fused to a normally trans-encoded tracrRNA, e.g., a single Cas9 guide RNA as described in Mali et al., Science 2013 Feb. 15; 339(6121):823-6, with a sequence at the 5′ end that is complementary to the target sequence, e.g., of 25-17, optionally 20 or fewer nucleotides (nts), e.g., 20, 19, 18, or 17 nts, preferably 17 or 18 nts, of the complementary strand to a target sequence immediately 5′ of a protospacer adjacent motif (PAM), e.g., NGG, NAG, or NNGG. In some embodiments, the single Cas9 guide RNA consists of the sequence:

(SEQ ID NO: 1)
(X₁₇₋₂₀)GUUUUAGAGCUAGAAAUAGCAAGUUAAAAUAAGGCUAGUCCG
(X_{N});
(SEQ ID NO: 2)
(X₁₇₋₂₀)GUUUUAGAGCUAUGCUGAAAAGCAUAGCAAGUUAAAAUAAGGC
UAGUCCGUUAUC(X_{N});
(SEQ ID NO: 3)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 4)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 5)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 6)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 7)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC;

wherein X₁₇₋₂₀ is the nucleotide sequence complementary to 17-20 consecutive nucleotides of the target sequence. DNAs encoding the single guide RNAs have been described previously in the literature (Jinek et al., Science. 337(6096):816-21 (2012) and Jinek et al., Elife. 2:e00471 (2013)).

The guide RNAs can include XN which can be any sequence, wherein N (in the RNA) can be 0-200, e.g., 0-100, 0-50, or 0-20, that does not interfere with the binding of the ribonucleic acid to Cas9.

In some embodiments, the guide RNA includes one or more Adenine (A) or Uracil (U) nucleotides on the 3′ end. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription.

Although some of the examples described herein utilize a single gRNA, the methods can also be used with dual gRNAs (e.g., the crRNA and tracrRNA found in naturally occurring systems). In this case, a single tracrRNA would be used in conjunction with multiple different crRNAs expressed using the present system, e.g., the following:
(X₁₇₋₂₀) GUUUUAGAGCUA (SEQ ID NO:102);
(X₁₇₋₂₀) GUUUUAGAGCUAUGCUGUUUUG (SEQ ID NO:103); or
(X₁₇₋₂₀) GUUUUAGAGCUAUGCU (SEQ ID NO:104); and a tracrRNA sequence.
   In this case, the crRNA is used as the guide RNA in the methods and molecules described herein, and the tracrRNA can be expressed from the same or a different DNA molecule. In some embodiments, the methods include contacting the cell with a tracrRNA comprising or consisting of the sequence
   GGAACCAUUCAAAACAGCAUAGCAAGUUAAAAUAAGGCUAGUCCGUUAUCA ACUUGAAAAAGUGGCACCGAGUCGGUGC (SEQ ID NO:8) or an active portion thereof (an active portion is one that retains the ability to form complexes with Cas9 or dCas9). In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Exemplary tracrRNA sequences in addition to SEQ ID NO:8 include the following:
   UAGCAAGUUAAAAUAAGGCUAGUCCGUUAUCAACUUGAAAAAGUGGCACCG AGUCGGUGC (SEQ ID NO:105) or an active portion thereof; or

AGCAUAGCAAGUUAAAAUAAGGCUAGUCCGUUAUCAACUUGAAAAAG UGGCACCGAGUCGGUGC (SEQ ID NO:106) or an active portion thereof.

In some embodiments when (X₁₇₋₂₀) GUUUUAGAGCUAUGCUGUUUUG (SEQ ID NO: 102) is used as a crRNA, the following tracrRNA is used:

GGAACCAUUCAAAACAGCAUAGCAAGUUAAAAUAAGGCUAGUCCGUUAUCA ACUUGAAAAAGUGGCACCGAGUCGGUGC (SEQ ID NO:8) or an active portion thereof.

In some embodiments when (X17-20) GUUUUAGAGCUA (SEQ ID NO:102) is used as a crRNA, the following tracrRNA is used:

UAGCAAGUUAAAAUAAGGCUAGUCCGUUAUCAACUUGAAAAAGUGGCACCG AGUCGGUGC (SEQ ID NO: 105) or an active portion thereof.

In some embodiments when (X17-20) GUUUUAGAGCUAUGCU (SEQ ID NO:104) is used as a crRNA, the following tracrRNA is used:

AGCAUAGCAAGUUAAAAUAAGGCUAGUCCGUUAUCAACUUGAAAAAGUGGC ACCGAGUCGGUGC (SEQ ID NO:106) or an active portion thereof.

In some embodiments, the gRNA is targeted to a site that is at least three or more mismatches different from any sequence in the rest of the genome in order to minimize off-target effects.

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity (Formula I).

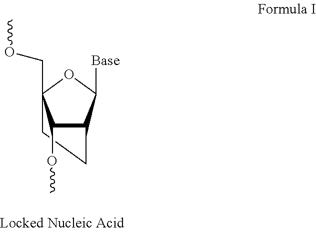

Thus in some embodiments, the tru-gRNAs disclosed herein may comprise one or more modified RNA oligonucleotides. For example, the truncated guide RNAs molecules described herein can have one, some or all of the region of the guideRNA complementary to the target sequence are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In other embodiments, one, some or all of the nucleotides of the tru-gRNA sequence may be modified, e.g., locked (2′-0-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In some embodiments, the single guide RNAs and/or crRNAs and/or tracrRNAs can include one or more Adenine (A) or Uracil (U) nucleotides on the 3′ end.

Existing Cas9-based RGNs use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the guide RNAs usable in the methods described herein can be hybrids, i.e., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA in a single gRNA system or alternatively might replace all of part of the crRNA and/or tracrRNA in a dual crRNA/tracrRNA system. Such a system that incorporates DNA into the complementarity region should more reliably target the intended genomic DNA sequences due to the general intolerance of DNA-DNA duplexes to mismatching compared to RNA-DNA duplexes. Methods for making such duplexes are known in the art, See, e.g., Barker et al., BMC Genomics. 2005 Apr. 22; 6:57; and Sugimoto et al., Biochemistry. 2000 Sep. 19; 39(37):11270-81.

In addition, in a system that uses separate crRNA and tracrRNA, one or both can be synthetic and include one or more modified (e.g., locked) nucleotides or deoxyribonucleotides.

In a cellular context, complexes of Cas9 with these synthetic gRNAs could be used to improve the genome-wide specificity of the CRISPR/Cas9 nuclease system.

The methods described can include expressing in a cell, or contacting the cell with, a Cas9 gRNA plus a fusion protein as described herein.

### Expression Systems

In order to use the fusion proteins and guide RNAs described herein, it may be desirable to express them from a nucleic acid that encodes them. This can be performed in a variety of ways. For example, a nucleic acid encoding a guide RNA or fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the guide RNA or fusion protein can also be cloned into an expression vector, for administration to a plant cell, animal cell, preferably a mammalian cell or a human cell, fungal cell, bacterial cell, or protozoan cell.

To obtain expression, a sequence encoding a guide RNA or fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the guide RNAs can include RNA Pol III promoters to drive expression of the guide RNAs, e.g., the H1, U6 or 7SK promoters. These human promoters allow for expression of gRNAs in mammalian cells following plasmid transfection. Alternatively, a T7 promoter may be used, e.g., for in vitro transcription, and the RNA can be transcribed in vitro and purified. Vectors suitable for the expression of short RNAs, e.g., siRNAs, shRNAs, or other small RNAs, can be used. Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

The present invention includes the vectors and cells comprising the vectors.

### EXAMPLES

The invention is further described in the following examples, which do not limit the scope of the invention described in the claims.

### Example 1. Engineering CRISPR/Cas Activator System

It was hypothesized that RNA-guided transcriptional activators could be created by fusing the strong synthetic VP64 activation domain (Beerli et al., Proc Natl Acad Sci USA 95, 14628-14633 (1998)) to the carboxy-terminus of the catalytically inactivated dCas9 protein (FIG. 1D).

To express guide RNAs (gRNAs) in human cells, a vector was engineered that would express the full length chimeric gRNA (a fusion of crRNA and tracrRNA originally described by Jinek et al. (Science 2012)) driven by a U6 promoter. Construction of the gRNA expression plasmids was performed as follows. Pairs of DNA oligonucleotides encoding the variable 20 nt gRNA targeting sequences were annealed together to generate short double-strand DNA fragments with 4 bp overhangs (Table 1).

TABLE 1
VEGFA and NTF3 gene target sites and associated
oligonucleotides used to construct gRNA
expression plasmids.

| gRNA | Target Site (including PAM) | SEQ ID NO: |
|---|---|---|
| V1 | GTGTGCAGACGGCAGTCACTAGG | 16. |
| V2 | GAGCAGCGTCTTCGAGAGTGAGG | 17. |
| V3 | GGTGAGTGAGTGTGTGCGTGTGG | 18. |
| V4 | GTTGGAGCGGGGAGAAGGCCAGG | 19. |
| V5 | GGGTGGGGGGAGTTTGCTCCTGG | 20. |
| V6 | GGCTTTGGAAAGGGGGTGGGGGG | 21. |
| V7 | GGGGCGGGGTCCCGGCGGGGCGG | 22. |
| V8 | GCTCGGAGGTCGTGGCGCTGGGG | 23. |
| V9 | GACTCACCGGCCAGGGCGCTCGG | 24. |
| V10 | GGCGCAGCGGTTAGGTGGACCGG | 25. |
| V11 | GGCGCATGGCTCCGCCCCGCCGG | 26. |
| V12 | GCCACGACCTCCGAGCTACCCGG | 27. |
| V13 | GCGGCGTGAGCCCTCCCCCTTGG | 28. |
| V14 | GGAGGCGGGGTGGAGGGGGTCGG | 29. |
| V15 | GGGCTCACGCCGCGCTCCGGCGG | 30. |
| V16 | GACCCCCTCCACCCCGCCTCCGG | 31. |
| N1 | GAGCGCGGAGCCATCTGGCCGGG | 32. |
| N2 | GCGCGGCGCGGAAGGGGTTAAGG | 33. |
| N3 | GCGGCGCGGCGCGGGCCGGCGGG | 34. |
| N4 | GCCGCGCCGCCCTCCCCCGCCGG | 35. |
| N5 | GCGGTTATAACCAGCCAACCCGG | 36. |
| N6 | GTGCGCGGAGCTGTTCGGAAGGG | 37. |
| gRNA | top oligo | SEQ ID NO: |
| V1 | ACACCGTGTGCAGACGGCAGTCACTG | 38. |
| V2 | ACACCGAGCAGCGTCTTCGAGAGTGG | 39. |
| V3 | ACACCGGTGAGTGAGTGTGTGCGTGG | 40. |
| V4 | ACACCGTTGGAGCGGGGAGAAGGCCG | 41. |
| V5 | ACACCGGGTGGGGGGAGTTTGCTCCG | 42. |
| V6 | ACACCGGCTTTGGAAAGGGGGTGGGG | 43. |
| V7 | ACACCGGGGCGGGGTCCCGGCGGGGG | 44. |
| V8 | ACACCGCTCGGAGGTCGTGGCGCTGG | 45. |
| V9 | ACACCGACTCACCGGCCAGGGCGCTG | 46. |
| V10 | ACACCGGCGCAGCGGTTAGGTGGACG | 47. |
| V11 | ACACCGGCGCATGGCTCCGCCCCGCG | 48. |
| V12 | ACACCGCCACGACCTCCGAGCTACCG | 49. |
| V13 | ACACCGCGGCGTGAGCCCTCCCCCTG | 50. |
| V14 | ACACCGGAGGCGGGGTGGAGGGGGTG | 51. |
| V15 | ACACCGGGCTCACGCCGCGCTCCGGG | 52. |
| V16 | ACACCGACCCCCTCCACCCCGCCTCG | 53. |
| N1 | ACACCGAGCGCGGAGCCATCTGGCCG | 54. |
| N2 | ACACCGCGCGGCGCGGAAGGGGTTAG | 55. |
| N3 | ACACCGCGGCGCGGCGCGGGCCGGCG | 56. |
| N4 | ACACCGCCGCGCCGCCCTCCCCCGCG | 57. |
| N5 | ACACCGCGGTTATAACCAGCCAACCG | 58. |
| N6 | ACACCGTGCGCGGAGCTGTTCGGAAG | 59. |
| gRNA | bottom oligo | SEQ ID NO: |
| V1 | AAAACAGTGACTGCCGTCTGCACACG | 60. |
| V2 | AAAACCACTCTCGAAGACGCTGCTCG | 61. |
| V3 | AAAACCACGCACACACTCACTCACCG | 62. |
| V4 | AAAACGGCCTTCTCCCCGCTCCAACG | 63. |
| V5 | AAAACGGAGCAAACTCCCCCCACCCG | 64. |
| V6 | AAAACCCCACCCCCTTTCCAAAGCCG | 65. |
| V7 | AAAACCCCCGCCGGGACCCCGCCCCG | 66. |
| V8 | AAAACCAGCGCCACGACCTCCGAGCG | 67. |
| V9 | AAAACAGCGCCCTGGCCGGTGAGTCG | 68. |
| V10 | AAAACGTCCACCTAACCGCTGCGCCG | 69. |
| V11 | AAAACGCGGGGCGGAGCCATGCGCCG | 70. |
| V12 | AAAACGGTAGCTCGGAGGTCGTGGCG | 71. |
| V13 | AAAACAGGGGGAGGGCTCACGCCGCG | 72. |
| V14 | AAAACACCCCCTCCACCCCGCCTCCG | 73. |
| V15 | AAAACCCGGAGCGCGGCGTGAGCCCG | 74. |
| V16 | AAAACGAGGCGGGGTGGAGGGGGTCG | 75. |
| N1 | AAAACGGCCAGATGGCTCCGCGCTCG | 76. |
| N2 | AAAACTAACCCCTTCCGCGCCGCGCG | 77. |
| N3 | AAAACGCCGGCCCGCGCCGCGCCGCG | 78. |
| N4 | AAAACGCGGGGGAGGGCGGCGCGGCG | 79. |
| N5 | AAAACGGTTGGCTGGTTATAACCGCG | 80. |
| N6 | AAAACTTCCGAACAGCTCCGCGCACG | 81. |

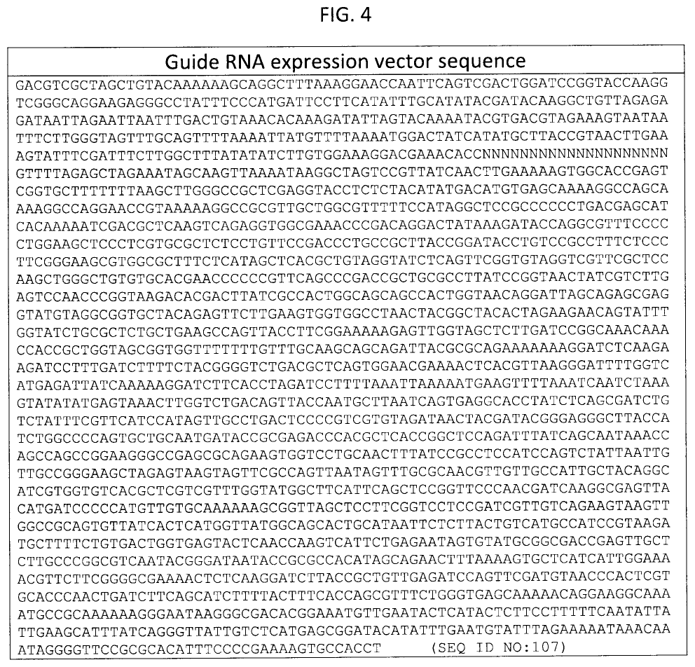

These fragments were ligated into BsmBI-digested plasmid pMLM3636 to yield DNA encoding a chimeric ˜102 nt single-chain guide RNA (Mali et al., Science. 2013 Feb. 15; 339 (6121):823-6; Hwang et al., Nat Biotechnol. 2013 March; 31(3):227-9) expressed by a human U6 promoter. The pMLM3636 plasmid and its full DNA sequence are available from Addgene. See FIG. 4.

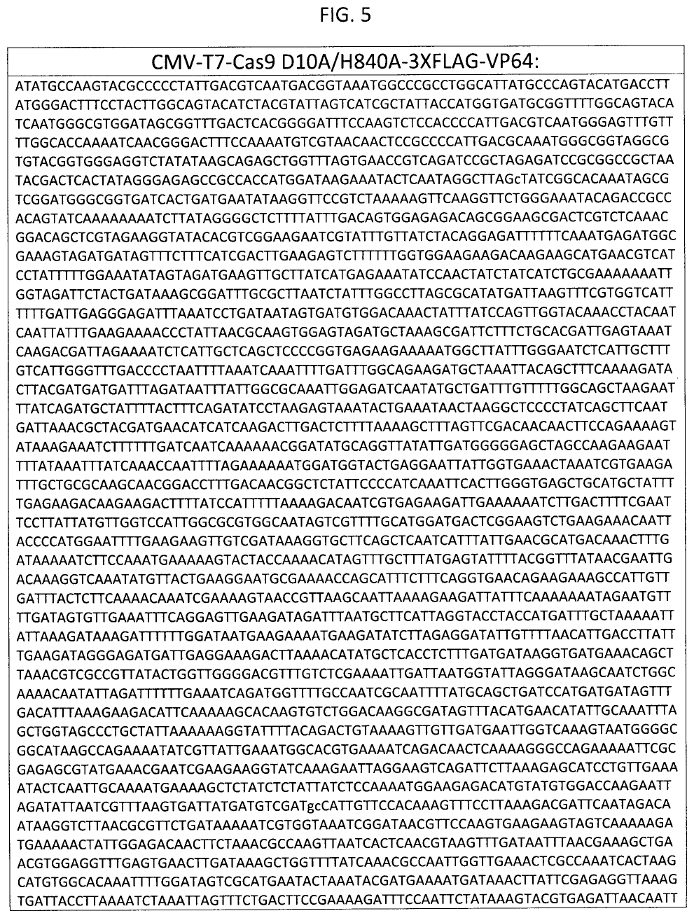

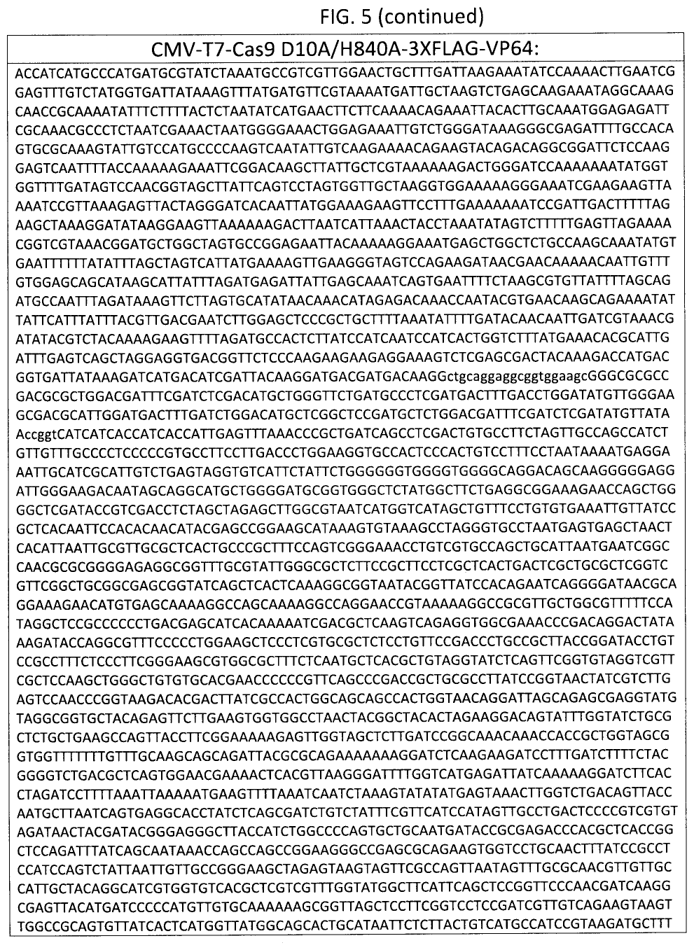

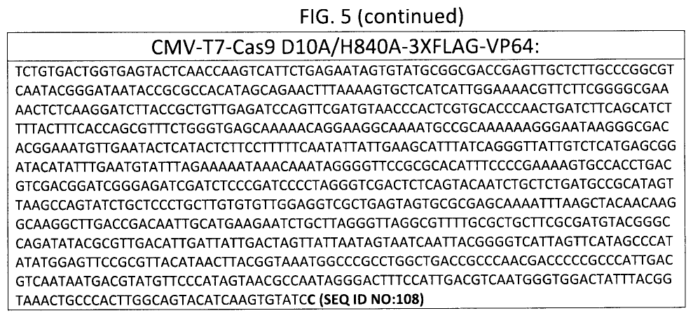

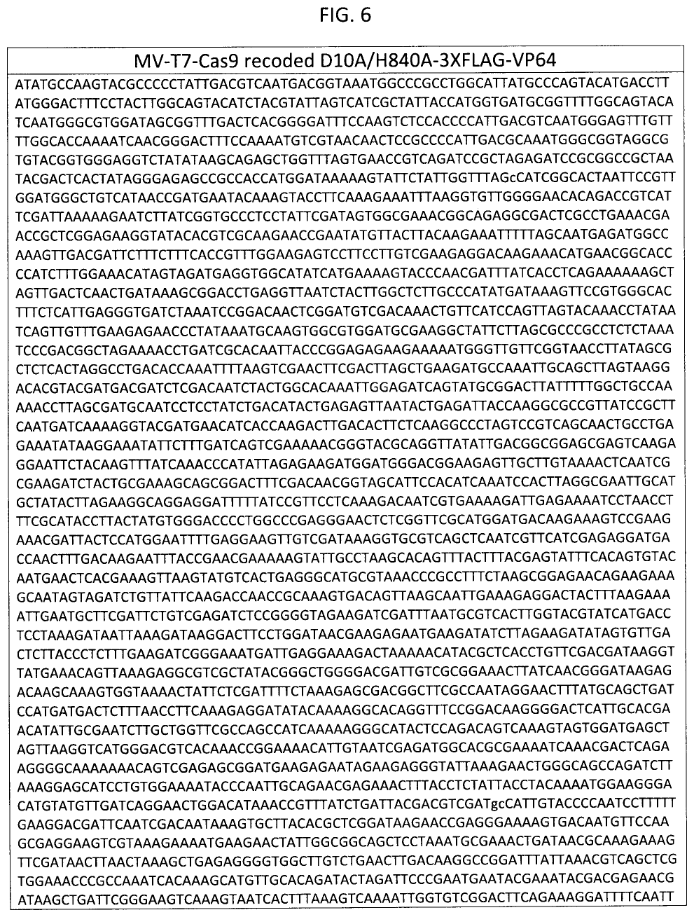

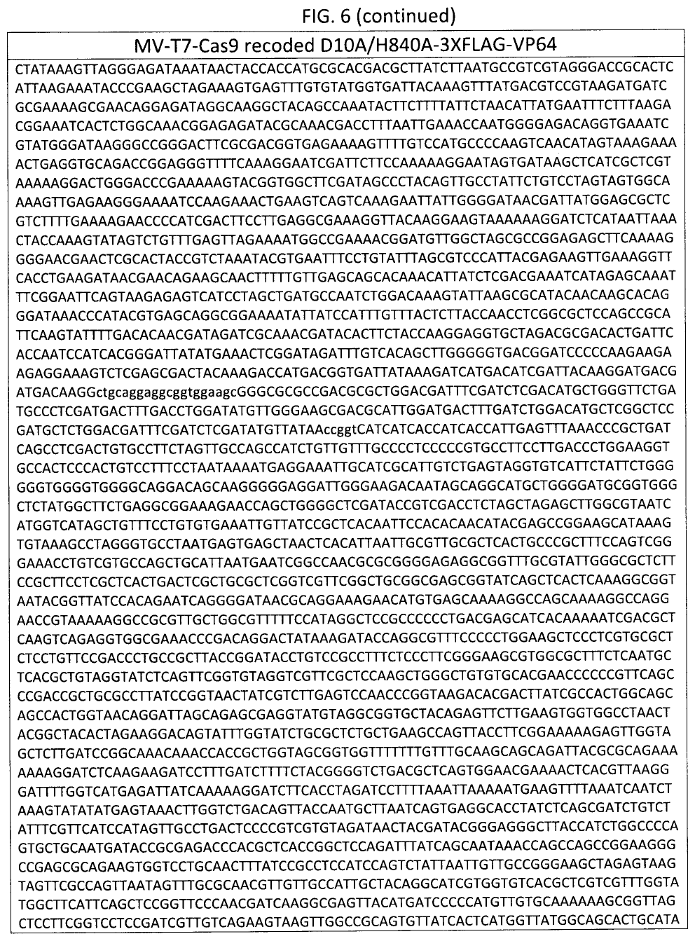

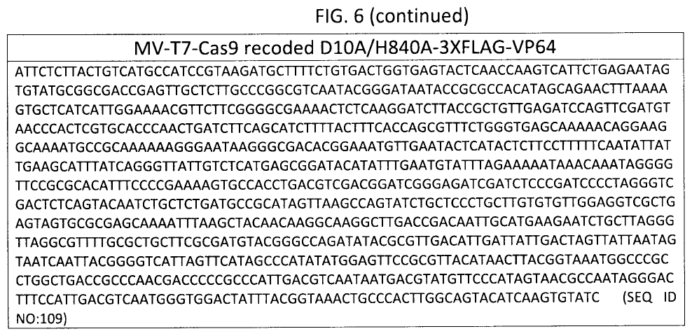

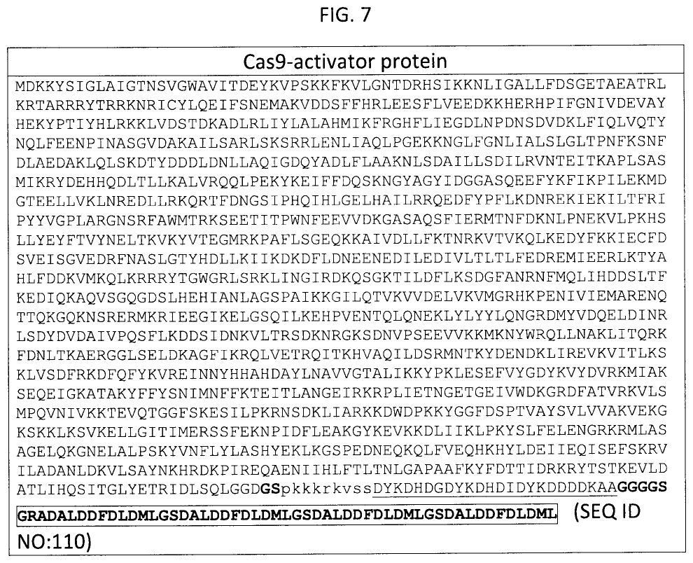

To engineer a Cas9-activator the D10A, H840A catalytic mutations (previously described in Jinek et al., 2012; and Qi et al., 2013) were introduced into either the wild-type or a codon-optimized Cas9 sequence (FIG. 5). These mutations render the Cas9 catalytically inactive so that it will no longer induce double-strand breaks. In one construct, a triple flag tag, nuclear localization signal and the VP64 activation domain were fused to the C-terminus of the inactive Cas9 (FIG. 6). Expression of this fusion protein was driven by the CMV promoter.

Construction of dCas-VP64 expression plasmids was performed as follows. DNA encoding the Cas9 nuclease harboring inactivating D10A/H840A mutations (dCas9) was amplified by PCR from plasmid pMJ841 (Addgene plasmid #39318) using primers that add a T7 promoter site 5′ to the start codon and a nuclear localization signal at the carboxy-terminal end of the Cas9 coding sequences and cloned into a plasmid containing a CMV promoter as previously described (Hwang et al., Nat Biotechnol 31, 227-229 (2013)) to yield plasmid pMLM3629. Oligonucleotides encoding a triple FLAG epitope were annealed and cloned into Xhol and PstI sites in plasmid pMLM3629 to generate plasmid pMLM3647 expressing dCas9 with a C-terminal flag FLAG tag. DNA sequence encoding a Gly₄Ser linker followed by the synthetic VP64 activation domain was introduced downstream of the FLAG-tagged dCas9 in plasmid pMLM3647 to yield plasmid pSL690. The D10A/H840A mutations were also introduced by QuikChange site-directed mutagenesis (Agilent) into plasmid pJDS247, which encodes a FLAG-tagged Cas9 sequence that has been codon optimized for expression in human cells, to yield plasmid pMLM3668. DNA sequence encoding the Gly₄Ser linker and the VP64 activation domain were then cloned into pMLM3668 to yield a codon-optimized dCas9-VP64 expression vector named pMLM3705.

Cell Culture, Transfection and ELISA Assays were performed as follows. Flp-In T-Rex 293 cells were maintained in Advanced DMEM supplemented with 10% FBS, 1% penstrep and 1% Glutamax (Invitrogen). Cells were transfected by Lipofectamine LTX (Invitrogen) according to manufacturer's instructions. Briefly, 160,000 293 cells were seeded in 24-well plates and transfected the following day with 250 ng gRNA plasmid, 250 ng Cas9-VP64 plasmid, 30ng pmaxGFP plasmid (Lonza), 0.5 ul Plus Reagent and 1.65 ul Lipofectamine LTX. Tissue culture media from transfected 293 cells was harvested 40 hours after transfection, and secreted VEGF-A protein assayed using R&D System's Human VEGF-A ELISA kit “Human VEGF Immunoassay.”

16 sgRNAs were constructed for target sequences within three DNase I hyper-sensitive sites (HSSs) located upstream, downstream or at the transcription start site of the human VEGFA gene in 293 cells (FIG. 1E).

Before testing the abilities of the 16 VEGFA-targeted gRNAs to recruit a novel dCas9-VP64 fusion protein, each of these gRNAs was first assessed for its ability to direct Cas9 nuclease to its intended target site in human 293 cells. For this purpose, gRNA and Cas9 expression vectors were transfected in a 1:3 ratio because previous optimization experiments demonstrated a high level of Cas9-induced DNA cleavage in U2OS cells using this ratio of plasmids.

Transfections of 293 cells were performed as described above for the dCas9-VP16 VEGFA experiments except that cells were transfected with 125 ng of plasmid encoding VEGFA-targeted gRNAs and 375 ng of plasmid encoding active Cas9 nuclease (pMLM3639). 40 hours post-transfection, genomic DNA was isolated using the QIAamp DNA Blood Mini kit (Qiagen) according to manufacturer's instructions. PCR amplification of the three different targeted regions in the VEGFA promoter was performed using Phusion Hot Start II high-fidelity DNA polymerase (NEB) with 3% DMSO and the following touchdown PCR cycle: 10 cycles of 98° C., 10 s; 72-62° C., −1° C./cycle, 15 s; 72° C., 30 s, followed by 25 cycles of 98° C., 10 s; 62° C., 15 s; 72° C., 30 s. The −500 region was amplified using primers oFYF434 (5′-TCCAGATGGCACATTGTCAG-3′ (SEQ ID NO: 82)) and oFYF435 (5′-AGGGAGCAGGAAAGTGAGGT-3′ (SEQ ID NO:83)). The region around the transcription start site was amplified using primers oFYF438 (5′-GCACGTAACCTCACTTTCCT-3′ (SEQ ID NO:84)) and oFYF439 (5′-CTTGCTACCTCTTTCCTCTTTCT-3′ (SEQ ID NO:85)). The +500 region was amplified using primers oFYF444 (5′-AGAGAAGTCGAGGAAGAGAGAG-3′ (SEQ ID NO:86)) and oFYF445 (5′-CAGCAGAAAGTTCATGGTTTCG-3′ (SEQ ID NO:87)). PCR products were purified using Ampure XP beads (Agencourt) and T7 Endonuclease I assays were performed and analyzed on a QIAXCEL capillary electrophoresis system as previously described (Reyon et al., Nat Biotech 30, 460-465 (2012)).

All 16 gRNAs were able to mediate the efficient introduction of Cas9 nuclease-induced indel mutations at their respective target sites as assessed using a previously described T7E1 genotyping assay (Table 2). Thus all 16 gRNAs can complex with Cas9 nuclease and direct its activity to specific target genomic sites in human cells.

TABLE 2
Frequencies of indel mutations induced by
VEGFA-targeted gRNAs and Cas9 nuclease

| | Mean Indel Mutation Frequency |
|---|---|
| gRNA | (%) ± SEM |
| V1 | 18.05 ± 0.47 |
| V2 | 41.48 ± 0.62 |
| V3 | 33.22 ± 1.05 |
| V4 | 16.97 ± 0.06 |
| V5 | 7.46 ± 0.50 |
| V6 | 16.99 ± 0.51 |
| V7 | 1.42 ± 0.11 |
| V8 | 34.07 ± 0.90 |
| V9 | 24.53 ± 1.40 |
| V10 | 35.65 ± 1.35 |
| V11 | 4.45 ± 0.22 |
| V12 | 23.95 ± 0.41 |
| V13 | 9.45 ± 0.74 |
| V14 | 12,17 ± 0.36 |
| V15 | 14.28 ± 0.54 |
| V16 | 18.82 ± 1.48 |

To test whether dCas9-VP64 protein could also be targeted to specific genomic sites in human cells by these same gRNAs, Enzyme-Linked Immunoblot Assays of VEGFA protein were performed as follows. Culture medium of Flp-In T-Rex HEK293 cells transfected with plasmids encoding VEGFA-targeted sgRNA and dCas9-VP64 was harvested 40 hours post-transfection and VEGFA protein expression was measured by ELISA as previously described (Maeder et al., Nat Methods 10, 243-245 (2013)). Fold-activation of VEGFA expression was calculated by dividing the concentration of VEGFA protein in media from cells in which both a sgRNA and dCas9-VP64 were expressed by the concentration of VEGFA protein in media from cells in which an off-target sgRNA (targeted to a sequence in the EGFP reporter gene) and dCas9-VP64 were expressed.

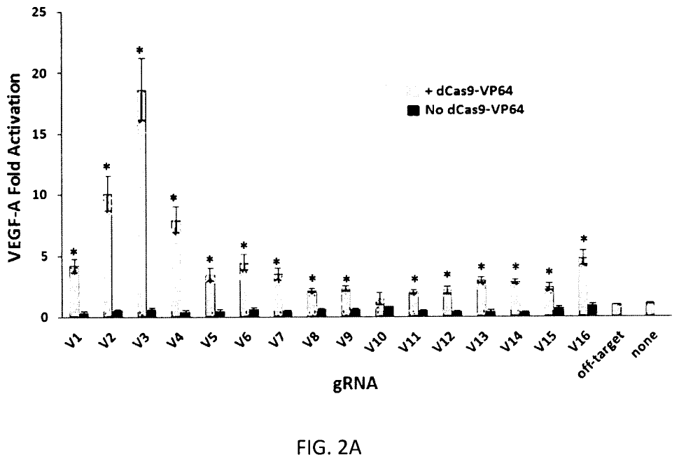

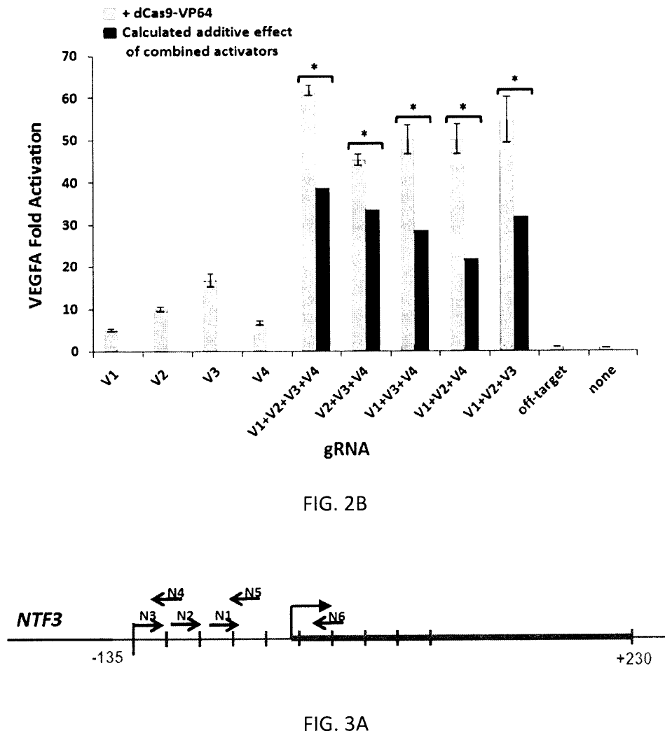

15 of the 16 gRNAs tested induced significant increases in VEGFA protein expression when co-expressed with dCas9-VP64 in human 293 cells (FIG. 2A). The magnitude of VEGFA induction observed ranged from two- to 18.7-fold-activation with a mean of five-fold-activation. Control experiments revealed that expression of each of the 16 gRNAs alone, dCas9-VP64 alone, and dCas9-VP64 together with an “off-target” gRNA designed to bind an EGFP reporter gene sequence all failed to induce elevated VEGFA expression (FIG. 2A), demonstrating that co-expression of a specific gRNA and the dCas9-VP64 protein are both required for promoter activation. Thus dCas9-VP64 is stably expressed and can be directed by gRNAs to activate transcription of specific genomic loci in human cells. The greatest increase in VEGFA was observed in cells transfected with gRNA3, which induced protein expression by 18.7-fold. Interestingly, the three best gRNAs, and 6 of the 9 gRNAs capable of inducing expression by 3-fold or more, target the −500 region (˜500bp upstream of the transcription start site).

Because in one aspect the system described herein uses variable gRNAs to recruit a common dCas9-VP64 activator fusion, one can envision that the expression of multiple guide RNAs in a single cell might enable multiplex or combinatorial activation of endogenous gene targets. To test this possibility, 293 cells were transfected with dCas9-VP64 expression plasmid together with expression plasmids for four gRNAs (V1, V2, V3, and V4) that each individually induced expression from the VEGFA promoter. Co-expression of all four gRNAs with dCas9-VP64 induced synergistic activation of VEGFA protein expression (i.e., a fold-activation greater than the expected additive effects of each individual activator) (FIG. 2B). In addition, various combinations of three of these four activators also activated the VEGFA promoter synergistically (FIG. 2B). Because synergistic activation of transcription is believed to result from the recruitment of multiple activator domains to a single promoter, multiple gRNA/dCas9-VP64 complexes are likely to be simultaneously binding to the VEGFA promoter in these experiments.

These experiments demonstrate that co-expression of a Cas9-HFD, e.g., a Cas9-activator protein (harboring the VP64 transcriptional activation domain) and a sgRNA with 20 nt of sequence complementarity to sites in the human VEGF-A promoter in human HEK293 cells can result in upregulation of VEGF-A expression. Increases in VEGF-A protein were measured by ELISA assay and it was found that individual gRNAs can function together with a Cas9-activator fusion protein to increase VEGF-A protein levels by up to ˜18-fold (FIG. 2A). Additionally, it was possible to achieve even greater increases in activation through transcriptional synergy by introducing multiple gRNAs targeting various sites in the same promoter together with Cas9-activator fusion proteins (FIG. 2B).

### Example 2. Engineering CRISPR/Cas Activator System Targeting the Endogenous Human NTF3 Gene

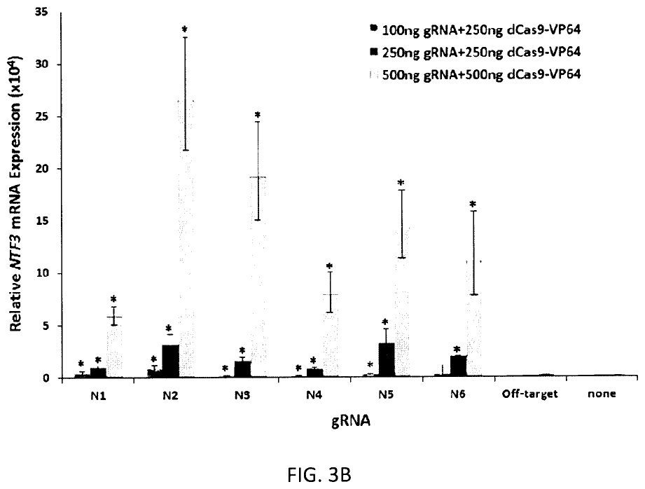

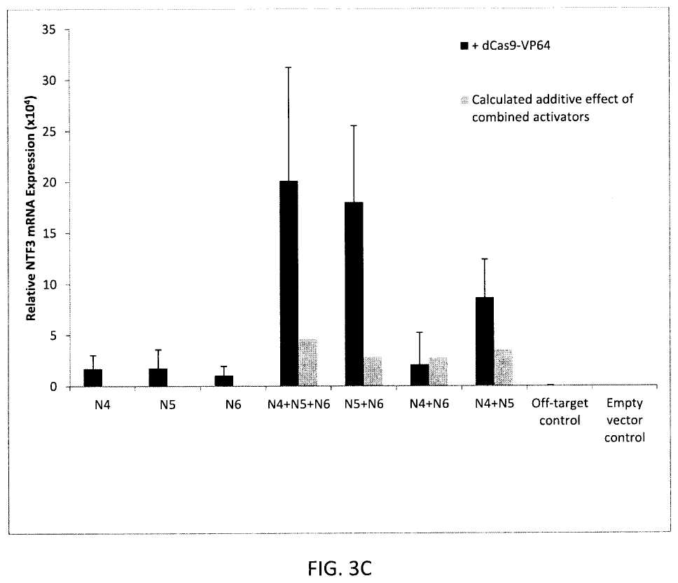

To extend the generality of the present findings, we tested whether the RNA-guided activator platform could be used to induce the expression of the human NTF3 gene. To do this, six sgRNAs were designed to a predicted DNase I hypersensitive site (HSS) in the human NTF3 promoter and plasmids expressing each of these gRNAs were co-transfected with a plasmid encoding dCas9-VP64 protein that had been codon optimized for human cell expression (FIG. 3A).

All six gRNAs tested induced significant increases in NTF3 transcript levels as detected by quantitative RT-PCR (FIG. 3B). Although fold-activation values for these six RNA-guided activators could not be accurately calculated (because basal levels of transcript were essentially undetectable), the mean levels of activated NTF3 mRNA expression varied over a four-fold range. Decreasing the amounts of gRNA and dCas9-VP64 expression plasmids transfected resulted in less activation of the NTF3 gene (FIG. 3B), demonstrating a clear dose-dependent effect.

In addition, 293 cells were co-transfected with dCas9-VP64 and NTF3-targeted gRNA expression plasmids alone and in single and double combinations. Relative expression of NTF3 mRNA was detected by quantitative RT-PCR and normalized to a GAPDH control (deltaCt×10⁴). In all of these experiments the amount of each individual gRNA expression plasmid used for transfection was the same. FIG. 3B shows that this multiplex gRNA expression induced synergistic activation of NTF3 mRNA expression by dCas9-VP64 protein.

### Example 3. Engineering CRISPR/Cas-MS2, -Csy4 and -Lambda N Fusion Systems—Creating Biological Tethers

Fusion proteins are made in which an MS2 coat protein, Csy4 nuclease (preferably catalytically inactive Csy4, e.g., the H29A mutant described in Haurwitz et al. 329(5997):1355-8 (2010)), or the lambda N are fused to the N- or C-terminus of the inactivated dCas9. MS2 and lambda N are bacteriophage proteins that bind to a specific RNA sequence, and thus can be used as adapters to tether to the dCas9 protein a heterologous RNA sequence tagged with the specific MS2 or lambda N RNA binding sequence. dCas9-MS2 fusions or dCas9-lambda N fusions are co-expressed with chimeric long non-coding RNAs (IncRNAs) fused to the MS2 or lambda N stem loop recognition sequence on either their 5′ or 3′ end. Chimeric Xist or chimeric RepA IncRNAs will be specifically recruited by the dCas9 fusions and the ability of this strategy to induce targeted silencing will be assayed by measuring target gene expression. The system will be optimized by testing various alterations to the coat proteins and chimeric RNAs. The N55K and deltaFG mutations to the MS2 coat protein have been previously demonstrated to prevent protein aggregation and increase affinity for the stem-loop RNA. Additionally, we will test the high-affinity C-loop RNA mutant reported to increase affinity for the MS2 coat protein. Exemplary sequences for the MS2 and lambda N proteins are given below; the MS2 functions as a dimer, therefore the MS2 protein can include a fused single chain dimer sequence.

1. Exemplary Sequences for Fusions of Single MS2 Coat Protein (wt, N55K or deltaFG) to the N-Terminus or C-Terminus of the dCas9

MS2 coat protein amino acid sequence:
(SEQ ID NO: 88)
MASNFTQFVLVDNGGTGDVTVAPSNFANGVAEWISSNSRSQAYKVTCSV
RQSSAQNRKYTIKVEVPKVATQTVGGVELPVAAWRSYLNMELTIPIFAT
NSDCELIVKAMQGLLKDGNPIPSAIAANSGIY
MS2 N55K:
(SEQ ID NO: 89)
MASNFTQFVLVDNGGTGDVTVAPSNFANGVAEWISSNSRSQAYKVTCSV
RQSSAQKRKYTIKVEVPKVATQTVGGVELPVAAWRSYLNMELTIPIFAT
NSDCELIVKAMQGLLKDGNPIPSAIAANSGIY
MS2deltaFG:
(SEQ ID NO: 90)
MASNFTQFVLVDNGGTGDVTVAPSNFANGIAEWISSNSRSQAYKVTCSV
RQSSAQNRKYTIKVEVPKGAWRSYLNMELTIPIFATNSDCELIVKAMQG
LLKDGNPIPSAIAANSGIY

2. Exemplary Sequences for Fusions of Fused Dimeric MS2 Coat Protein (wt, N55K or deltaFG) to the N-Terminus or C-Terminus of dCas9

Dimeric MS2 coat protein:
(SEQ ID NO: 91
MASNFTQFVLVDNGGTGDVTVAPSNFANGVAEWISSNSRSQAYKVTCSV
RQSSAQNRKYTIKVEVPKVATQTVGGVELPVAAWRSYLNMELTIPIFAT
NSDCELIVKAMQGLLKDGNPIPSAIAANSGLYGAMASNFTQFVLVDNGG
TGDVTVAPSNFANGVAEWISSNSRSQAYKVTCSVRQSSAQNRKYTIKVE
VPKVATQTVGGVELPVAAWRSYLNMELTIPIFATNSDCELIVKAMQGLL
KDGNPIPSAIAANSLIN
(SEQ ID NO: 92)
MASNFTQFVLVDNGGTGDVTVAPSNFANGVAEWISSNSRSQAYKVTCSV
RQSSAQKRKYTIKVEVPKVATQTVGGVELPVAAWRSYLNMELTIPIFAT
NSDCELIVKAMQGLLKDGNPIPSAIAANSGLYGAMASNFTQFVLVDNGG
TGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLL
KDGNPIPSAIAANSLIN
Dimeric MS2deltaFG:
(SEQ ID NO: 93)
MASNFTQFVLVDNGGTGDVTVAPSNFANGVAEWISSNSRSQAYKVTCSV
RQSSAQKRKYTIKVEVPKGAWRSYLNMELTIPIFATNSDCELIVKAMQG
LLKDGNPIPSAIAANSGLYGAMASNFTQFVLVDNGGTGDVTVAPSNFAN
GVAEWISSNSRSQAYKVTCSVRQSSAQKRKYTIKVEVPKGAWRSYLNME
LTIPIFATNSDCELIVKAMQGLLKDGNPIPSAIAANSLIN

3. Exemplary Sequences for Fusions of Lambda N to N-Terminus or C-Terminus of dCas9

Lambda N amino acid sequence:
(SEQ ID NO: 94)
MDAQTRRRERRAEKQAQWKAAN
or
(SEQ ID NO: 95)
MDAQTRRRERRAEKQAQWKAANPLLVGVSAKPVNRPILSLNRKPKSRVE
SALNPIDLTVLAEYHKQIESNLQRIERKNQRTWYSKPGERGITCSGRQK
IKGKSIPLI

4. Exemplary Sequence for Fusions of Csy4 to N-Terminus or C-Terminus of dCas9

Exemplary sequences for Cys4 are given in Haurwitz et al. 329(5997):1355-8 (2010), e.g., the inactivated form.

The constructs are expressed in cells also expressing a regulatory RNA, e.g., a long non-coding RNA (IncRNA) such as HOTAIR, HOTTIP, XIST or XIST RepA, that has been fused with the cognate stem-loop recognition sequence for the lambda N or MS2 on either its 5′ or 3′ end. The wild type and high-affinity sequences for MS2 are AAACAUGAGGAUUACCCAUGUCG (SEQ ID NO:96) and AAACAUGAGGAUCACCCAUGUCG (SEQ ID NO:97), respectively (see Keryer-Bibens et al., supra, FIG. 2); the nutL and nutR BoxB sequences to which lambda N binds are GCCCUGAAGAAGGGC (SEQ ID NO:98) and GCCCUGAAAAAGGGC (SEQ ID NO: 99), respectively. The sequence to which Csy4 binds is GTTCACTGCCGTATAGGCAG (truncated 20 nt) (SEQ ID NO:100) or GUUCACUGCCGUAUAGGCAGCUAAGAAA (SEQ ID NO:101).

The binding of the dCas9/MS2 to a target site in a cell expressing an MS2-binding sequence tagged IncRNA recruits that IncRNA to the dCas9 binding site; where the IncRNA is a repressor, e.g., XIST, genes near the dCas9 binding site are repressed. Similarly, binding of the dCas9/lambdaN to a target site in a cell expressing an lambdaN-binding sequence tagged IncRNA recruits that IncRNA to the dCas9 binding site.

### Example 4. Engineering CRISPR/Cas-HP1 Fusion Systems-Sequence-Specific Silencing

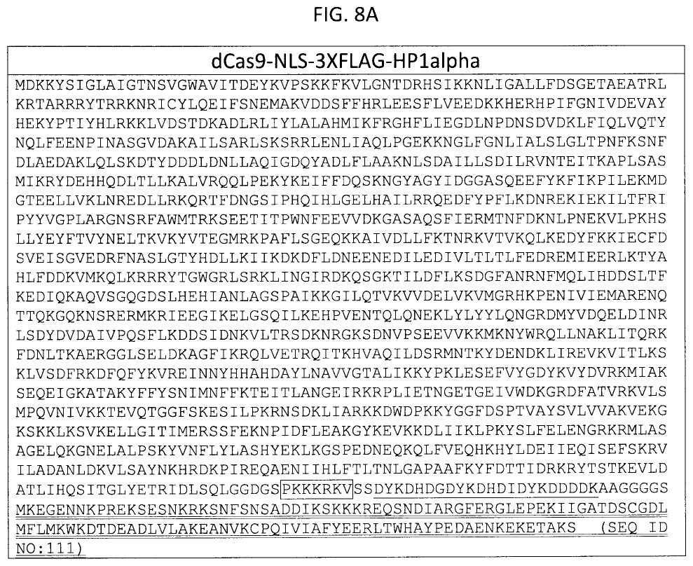

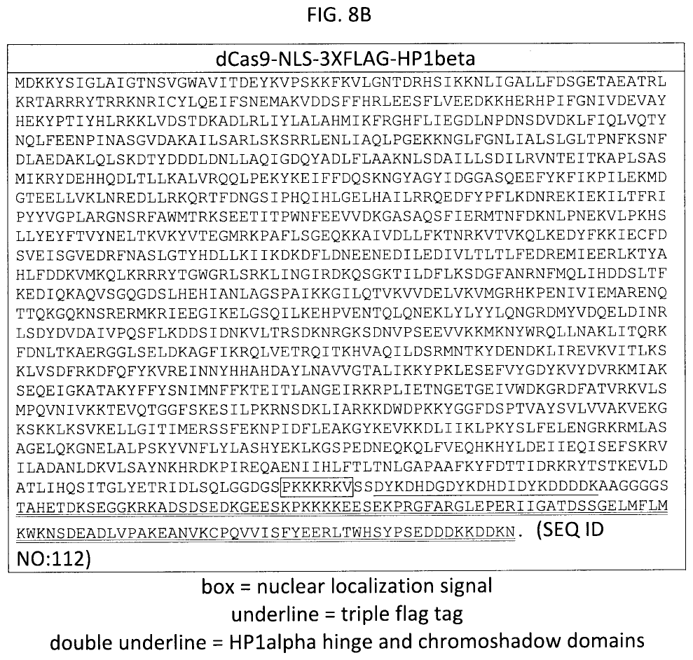

The dCas9 fusion proteins described herein can also be used to target silencing domains, e.g., Heterochromatin Protein 1 (HP1, also known as swi6), e.g., HP1α or HP1β. Truncated versions of HP1α or HP1β in which the chromodomain has been removed can be targeted to specific loci to induce heterochromatin formation and gene silencing. Exemplary sequences of truncated HP1 fused to dCas9 are shown in FIGS. 8A-8B. The HP1 sequences can be fused to the N- or C-terminus of the inactivated dCas9 as described above.

### Example 5. Engineering CRISPR/Cas-TET Fusion Systems-Sequence-Specific Demethylation

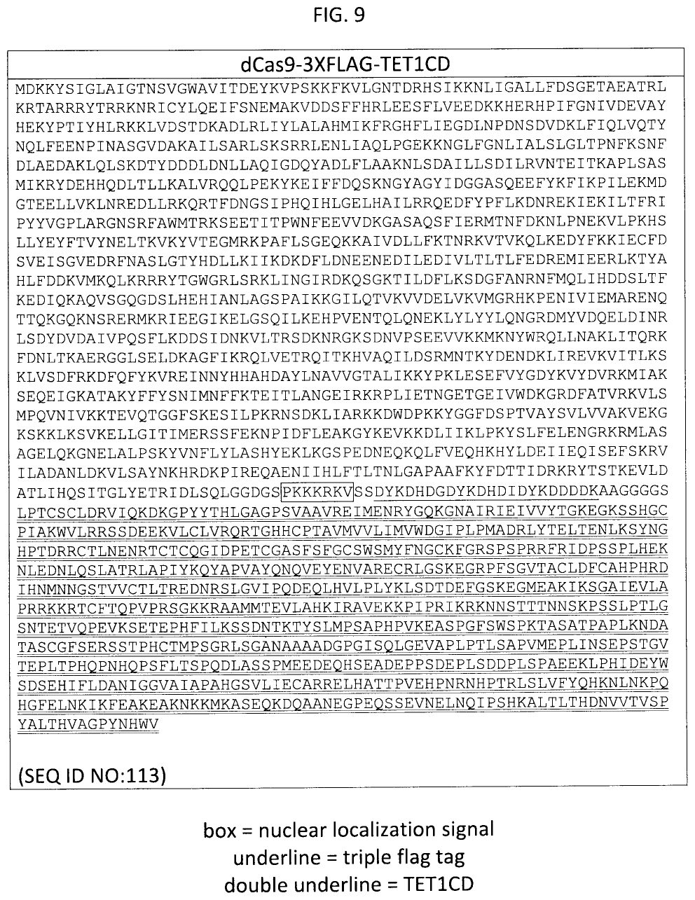

The dCas9 fusion proteins described herein can also be used to target enzymes that modify the methylation state of DNA (e.g., DNA methyltransferase (DNMT) or TET proteins). Truncated versions of TETI can be targeted to specific loci to catalyze DNA demethylation. Exemplary sequences of truncated TETI fused to dCas9 are shown in FIG. 9. The TETI sequence can be fused to the N- or C-terminus of the inactivated dCas9 as described above.

### Example 6. Engineering Optimized CRISPR/Cas-VP64 Fusions

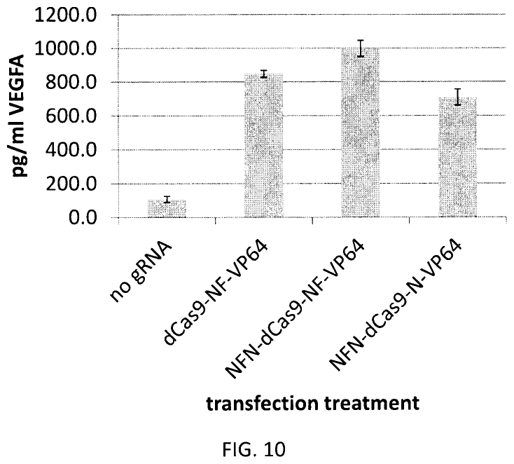

The activities of dCas9-based transcription activators harboring the VP64 activation domain were optimized by varying the number and position of the nuclear localization signal(s) (NLS) and 3×FLAG-tags within these fusions (FIG. 10). dCas9-VP64 fusions that contain both an N-terminal NLS and an NLS that lies between the dCas9 and VP64 sequences consistently induce higher levels of target gene activation, perhaps resulting from enhanced nuclear localization of the activator (FIG. 10). Furthermore, even greater levels of activation were observed when a 3×FLAG tag was placed between the C-terminal end of dCas9 and the N-terminal end of VP64. The 3×FLAG tag may act as an artificial linker, providing necessary spacing between dCas9 and VP64 and perhaps allowing for better folding of the VP64 domain (that may not be possible when constrained near dCas9) or better recognition of VP64 by transcriptional mediator complexes that recruit RNA polymerase II. Alternatively, the negatively charged 3×FLAG tag might also function as a fortuitous transcriptional activation domain, enhancing the effects of the VP64 domain.

### Example 7. Optimized CatalyticallyCatlytically Inactive Cas9 Proteins (dCas9)

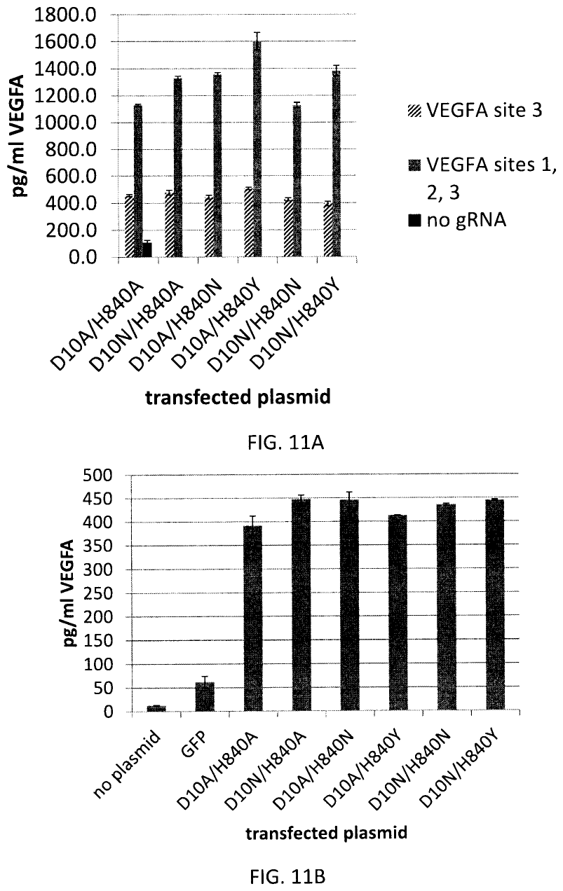

Additional optimization of the activities of dCas9-VP64 activators was performed by changing the nature of the inactivating mutations that abolish the nuclease activity of Cas9 in the dCas9 domain (FIG. 11A-B). In published studies to date, the catalytic residues D10 and H840 were mutated to alanine (D10A and H840A) to disrupt the active site networks that mediate the hydrolysis of DNA. It was hypothesized that alanine substitutions at these positions might result in destabilization of dCas9 and therefore suboptimal activity. Therefore, more structurally conservative substitutions at D10 or H840 (for example, to asparagine or tyrosine residues: D1ON, H840N, and H840Y) were tested to see if they might lead to greater gene activation by dCas9-VP64 fusions bearing these different mutations. When dCas9-VP64 variants bearing these variant substitutions were co-transfected into HEK293 cells with three gRNAs targeting upstream regions of the endogenous human VEGFA gene, greater VEGFA protein expression was observed for all but one of these variants (FIG. 11A). However, this effect was not as significant when the dCas9-VP64 variants were co-transfected with only one of these gRNAs (FIG. 11A), or when transfected into a HEK293 derivative cell-line that expresses a single VEGFA-targeted gRNA (FIG. 11B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-25. (canceled)

26. A mammalian cell expressing a fusion protein comprising a catalytically inactive S. pyogenes Cas9 protein linked to a heterologous functional domain, wherein the catalytically inactive Cas9 protein comprises mutations at D10 and H840.

27. The mammalian cell of claim 26, wherein the heterologous functional domain is a transcriptional activation domain.

28. The mammalian cell of claim 27, wherein the transcriptional activation domain is from VP64 or NF-κB p65.

29. The mammalian cell of claim 26, wherein the heterologous functional domain is a transcriptional silencer.

30. The mammalian cell of claim 29, wherein the transcriptional silencer is a Heterochromatin Protein 1 (HP1) domain.

31. The mammalian cell of claim 26, wherein the heterologous functional domain is a transcriptional repression domain.

32. The mammalian cell of claim 31, wherein the transcriptional repression domain is a Krueppel-associated box (KRAB) domain, an ERF repressor domain (ERD), or a mSin3A interaction domain (SID).

33. The mammalian cell of claim 26, wherein the heterologous functional domain is linked to the N-terminus or the C-terminus of the catalytically inactive Cas9 protein, with an optional intervening linker, wherein the linker does not interfere with activity of the fusion protein.

34. The mammalian cell of claim 26, further comprising one or both of a nuclear localization sequence and one or more epitope tags on the N-terminus, the C-terminus, or in between the catalytically inactive Cas9 protein and the heterologous functional domain, optionally with one or more intervening linkers.

35. The mammalian cell of claim 34, wherein the one or more epitope tags is selected from the group consisting of c-myc, 6His, and FLAG tags.

36. The mammalian cell of claim 26, wherein expressing the fusion protein in the mammalian cell comprises contacting the mammalian cell with an expression vector comprising a promoter operably linked to a nucleic acid encoding the fusion protein.


SEQUENCE LISTING (113 sequences)

SEQ ID NO: 1 - RNA, 262 nt, synthetic construct
  source 1..262: mol_type = other RNA
nnnnnnnnnnnnnnnnnnnngttttagagctagaaatagcaagttaaaataaggctagtccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 2 - RNA, 275 nt, synthetic construct
  source 1..275: mol_type = other RNA
nnnnnnnnnnnnnnnnnnnngttttagagctatgctgaaaagcatagcaagttaaaataaggctagtccgttatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 3 - RNA, 287 nt, synthetic construct
  source 1..287: mol_type = other RNA
nnnnnnnnnnnnnnnnnnnngttttagagctatgctgttttggaaacaaaacagcatagcaagttaaaataaggctagtccgttatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 4 - RNA, 296 nt, synthetic construct
  source 1..296: mol_type = other RNA
nnnnnnnnnnnnnnnnnnnngttttagagctagaaatagcaagttaaaataaggctagtccgttatcaacttgaaaaagtggcaccgagtcggtgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 5 - RNA, 96 nt, synthetic construct
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 6 - RNA, 106 nt, synthetic construct
  source 1..106: mol_type = other RNA
nnnnnnnnnnnnnnnnnnnngttttagagctatgctggaaacagcatagcaagtttaaataaggctagtccgttatcaacttgaaaaagtggcaccgagtcggtgc

SEQ ID NO: 7 - RNA, 106 nt, synthetic construct
  source 1..106: mol_type = other RNA
nnnnnnnnnnnnnnnnnnnngtttaagagctatgctggaaacagcatagcaagtttaaataaggctagtccgttatcaacttgaaaaagtggcaccgagtcggtgc

SEQ ID NO: 8 - RNA, 79 nt, synthetic construct
  source 1..79: mol_type = other RNA
ggaaccattcaaaacagcatagcaagttaaaataaggctagtccgttatcaacttgaaaaagtggcaccgagtcggtgc

SEQ ID NO: 9 - RNA, 62 nt, synthetic construct
  source 1..62: mol_type = other RNA
ggagcgagcggagcggtacagttttagagctagaaatagcaagttaaaataaggctagtccg

SEQ ID NO: 10 - RNA, 100 nt, synthetic construct
  source 1..100: mol_type = other RNA
ggagcgagcggagcggtacagttttagagctagaaatagcaagttaaaataaggctagtccgttatcaacttgaaaaagtggcaccgagtcggtgctttt

SEQ ID NO: 11 - protein, 8 aa, synthetic construct
PKKKRKVS

SEQ ID NO: 12 - protein, 50 aa, synthetic construct
DALDDFDLDMLGSDALDDFDLDMLGSDALDDFDLDMLGSDALDDFDLDML

SEQ ID NO: 13 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 4 aa, synthetic construct
GGGS

SEQ ID NO: 15 - protein, 5 aa, synthetic construct
GGGGS

SEQ ID NO: 16 - DNA, 23 bp, synthetic construct
  source 1..23: mol_type = other DNA
gtgtgcagacggcagtcactagg

SEQ ID NO: 17 - DNA, 23 bp, synthetic construct
  source 1..23: mol_type = other DNA
gagcagcgtcttcgagagtgagg

SEQ ID NO: 18 - DNA, 23 bp, synthetic construct
  source 1..23: mol_type = other DNA
ggtgagtgagtgtgtgcgtgtgg

SEQ ID NO: 19 - DNA, 23 bp, synthetic construct
  source 1..23: mol_type = other DNA
gttggagcggggagaaggccagg

SEQ ID NO: 20 - DNA, 23 bp, synthetic construct
  source 1..23: mol_type = other DNA
gggtggggggagtttgctcctgg

SEQ ID NO: 21 - DNA, 23 bp, synthetic construct
  source 1..23: mol_type = other DNA
ggctttggaaagggggtgggggg

SEQ ID NO: 22 - DNA, 23 bp, synthetic construct
  source 1..23: mol_type = other DNA
ggggcggggtcccggcggggcgg

SEQ ID NO: 23 - DNA, 23 bp, synthetic construct
  source 1..23: mol_type = other DNA
gctcggaggtcgtggcgctgggg

SEQ ID NO: 24 - DNA, 23 bp, synthetic construct
  source 1..23: mol_type = other DNA
gactcaccggccagggcgctcgg

SEQ ID NO: 25 - DNA, 23 bp, synthetic construct
  source 1..23: mol_type = other DNA
ggcgcagcggttaggtggaccgg

SEQ ID NO: 26 - DNA, 23 bp, synthetic construct
  source 1..23: mol_type = other DNA
ggcgcatggctccgccccgccgg

SEQ ID NO: 27 - DNA, 23 bp, synthetic construct
  source 1..23: mol_type = other DNA
gccacgacctccgagctacccgg

SEQ ID NO: 28 - DNA, 23 bp, synthetic construct
  source 1..23: mol_type = other DNA
gcggcgtgagccctcccccttgg

SEQ ID NO: 29 - DNA, 23 bp, synthetic construct
  source 1..23: mol_type = other DNA
ggaggcggggtggagggggtcgg

SEQ ID NO: 30 - DNA, 23 bp, synthetic construct
  source 1..23: mol_type = other DNA
gggctcacgccgcgctccggcgg

SEQ ID NO: 31 - DNA, 23 bp, synthetic construct
  source 1..23: mol_type = other DNA
gaccccctccaccccgcctccgg

SEQ ID NO: 32 - DNA, 23 bp, synthetic construct
  source 1..23: mol_type = other DNA
gagcgcggagccatctggccggg

SEQ ID NO: 33 - DNA, 23 bp, synthetic construct
  source 1..23: mol_type = other DNA
gcgcggcgcggaaggggttaagg

SEQ ID NO: 34 - DNA, 23 bp, synthetic construct
  source 1..23: mol_type = other DNA
gcggcgcggcgcgggccggcggg

SEQ ID NO: 35 - DNA, 23 bp, synthetic construct
  source 1..23: mol_type = other DNA
gccgcgccgccctcccccgccgg

SEQ ID NO: 36 - DNA, 23 bp, synthetic construct
  source 1..23: mol_type = other DNA
gcggttataaccagccaacccgg

SEQ ID NO: 37 - DNA, 23 bp, synthetic construct
  source 1..23: mol_type = other DNA
gtgcgcggagctgttcggaaggg

SEQ ID NO: 38 - DNA, 26 bp, synthetic construct
  source 1..26: mol_type = other DNA
acaccgtgtgcagacggcagtcactg

SEQ ID NO: 39 - DNA, 26 bp, synthetic construct
  source 1..26: mol_type = other DNA
acaccgagcagcgtcttcgagagtgg

SEQ ID NO: 40 - DNA, 26 bp, synthetic construct
  source 1..26: mol_type = other DNA
acaccggtgagtgagtgtgtgcgtgg

SEQ ID NO: 41 - DNA, 26 bp, synthetic construct
  source 1..26: mol_type = other DNA
acaccgttggagcggggagaaggccg

SEQ ID NO: 42 - DNA, 26 bp, synthetic construct
  source 1..26: mol_type = other DNA
acaccgggtggggggagtttgctccg

SEQ ID NO: 43 - DNA, 26 bp, synthetic construct
  source 1..26: mol_type = other DNA
acaccggctttggaaagggggtgggg

SEQ ID NO: 44 - DNA, 26 bp, synthetic construct
  source 1..26: mol_type = other DNA
acaccggggcggggtcccggcggggg

SEQ ID NO: 45 - DNA, 26 bp, synthetic construct
  source 1..26: mol_type = other DNA
acaccgctcggaggtcgtggcgctgg

SEQ ID NO: 46 - DNA, 26 bp, synthetic construct
  source 1..26: mol_type = other DNA
acaccgactcaccggccagggcgctg

SEQ ID NO: 47 - DNA, 26 bp, synthetic construct
  source 1..26: mol_type = other DNA
acaccggcgcagcggttaggtggacg

SEQ ID NO: 48 - DNA, 26 bp, synthetic construct
  source 1..26: mol_type = other DNA
acaccggcgcatggctccgccccgcg

SEQ ID NO: 49 - DNA, 26 bp, synthetic construct
  source 1..26: mol_type = other DNA
acaccgccacgacctccgagctaccg

SEQ ID NO: 50 - DNA, 26 bp, synthetic construct
  source 1..26: mol_type = other DNA
acaccgcggcgtgagccctccccctg

SEQ ID NO: 51 - DNA, 26 bp, synthetic construct
  source 1..26: mol_type = other DNA
acaccggaggcggggtggagggggtg

SEQ ID NO: 52 - DNA, 26 bp, synthetic construct
  source 1..26: mol_type = other DNA
acaccgggctcacgccgcgctccggg

SEQ ID NO: 53 - DNA, 26 bp, synthetic construct
  source 1..26: mol_type = other DNA
acaccgaccccctccaccccgcctcg

SEQ ID NO: 54 - DNA, 26 bp, synthetic construct
  source 1..26: mol_type = other DNA
acaccgagcgcggagccatctggccg

SEQ ID NO: 55 - DNA, 26 bp, synthetic construct
  source 1..26: mol_type = other DNA
acaccgcgcggcgcggaaggggttag

SEQ ID NO: 56 - DNA, 26 bp, synthetic construct
  source 1..26: mol_type = other DNA
acaccgcggcgcggcgcgggccggcg

SEQ ID NO: 57 - DNA, 26 bp, synthetic construct
  source 1..26: mol_type = other DNA
acaccgccgcgccgccctcccccgcg

SEQ ID NO: 58 - DNA, 26 bp, synthetic construct
  source 1..26: mol_type = other DNA
acaccgcggttataaccagccaaccg

SEQ ID NO: 59 - DNA, 26 bp, synthetic construct
  source 1..26: mol_type = other DNA
acaccgtgcgcggagctgttcggaag

SEQ ID NO: 60 - DNA, 26 bp, synthetic construct
  source 1..26: mol_type = other DNA
aaaacagtgactgccgtctgcacacg

SEQ ID NO: 61 - DNA, 26 bp, synthetic construct
  source 1..26: mol_type = other DNA
aaaaccactctcgaagacgctgctcg

SEQ ID NO: 62 - DNA, 26 bp, synthetic construct
  source 1..26: mol_type = other DNA
aaaaccacgcacacactcactcaccg

SEQ ID NO: 63 - DNA, 26 bp, synthetic construct
  source 1..26: mol_type = other DNA
aaaacggccttctccccgctccaacg

SEQ ID NO: 64 - DNA, 26 bp, synthetic construct
  source 1..26: mol_type = other DNA
aaaacggagcaaactccccccacccg

SEQ ID NO: 65 - DNA, 26 bp, synthetic construct
  source 1..26: mol_type = other DNA
aaaaccccaccccctttccaaagccg

SEQ ID NO: 66 - DNA, 26 bp, synthetic construct
  source 1..26: mol_type = other DNA
aaaacccccgccgggaccccgccccg

SEQ ID NO: 67 - DNA, 26 bp, synthetic construct
  source 1..26: mol_type = other DNA
aaaaccagcgccacgacctccgagcg

SEQ ID NO: 68 - DNA, 26 bp, synthetic construct
  source 1..26: mol_type = other DNA
aaaacagcgccctggccggtgagtcg

SEQ ID NO: 69 - DNA, 26 bp, synthetic construct
  source 1..26: mol_type = other DNA
aaaacgtccacctaaccgctgcgccg

SEQ ID NO: 70 - DNA, 26 bp, synthetic construct
  source 1..26: mol_type = other DNA
aaaacgcggggcggagccatgcgccg

SEQ ID NO: 71 - DNA, 26 bp, synthetic construct
  source 1..26: mol_type = other DNA
aaaacggtagctcggaggtcgtggcg

SEQ ID NO: 72 - DNA, 26 bp, synthetic construct
  source 1..26: mol_type = other DNA
aaaacagggggagggctcacgccgcg

SEQ ID NO: 73 - DNA, 26 bp, synthetic construct
  source 1..26: mol_type = other DNA
aaaacaccccctccaccccgcctccg

SEQ ID NO: 74 - DNA, 26 bp, synthetic construct
  source 1..26: mol_type = other DNA
aaaacccggagcgcggcgtgagcccg

SEQ ID NO: 75 - DNA, 26 bp, synthetic construct
  source 1..26: mol_type = other DNA
aaaacgaggcggggtggagggggtcg

SEQ ID NO: 76 - DNA, 26 bp, synthetic construct
  source 1..26: mol_type = other DNA
aaaacggccagatggctccgcgctcg

SEQ ID NO: 77 - DNA, 26 bp, synthetic construct
  source 1..26: mol_type = other DNA
aaaactaaccccttccgcgccgcgcg

SEQ ID NO: 78 - DNA, 26 bp, synthetic construct
  source 1..26: mol_type = other DNA
aaaacgccggcccgcgccgcgccgcg

SEQ ID NO: 79 - DNA, 26 bp, synthetic construct
  source 1..26: mol_type = other DNA
aaaacgcgggggagggcggcgcggcg

SEQ ID NO: 80 - DNA, 26 bp, synthetic construct
  source 1..26: mol_type = other DNA
aaaacggttggctggttataaccgcg

SEQ ID NO: 81 - DNA, 26 bp, synthetic construct
  source 1..26: mol_type = other DNA
aaaacttccgaacagctccgcgcacg

SEQ ID NO: 82 - DNA, 20 bp, synthetic construct
  source 1..20: mol_type = other DNA
tccagatggcacattgtcag

SEQ ID NO: 83 - DNA, 20 bp, synthetic construct
  source 1..20: mol_type = other DNA
agggagcaggaaagtgaggt

SEQ ID NO: 84 - DNA, 20 bp, synthetic construct
  source 1..20: mol_type = other DNA
gcacgtaacctcactttcct

SEQ ID NO: 85 - DNA, 23 bp, synthetic construct
  source 1..23: mol_type = other DNA
cttgctacctctttcctctttct

SEQ ID NO: 86 - DNA, 22 bp, synthetic construct
  source 1..22: mol_type = other DNA
agagaagtcgaggaagagagag

SEQ ID NO: 87 - DNA, 22 bp, synthetic construct
  source 1..22: mol_type = other DNA
cagcagaaagttcatggtttcg

SEQ ID NO: 88 - protein, 130 aa, Enterobacteria phage lambda
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 89 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 90 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 91 - protein, 262 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSLIN

SEQ ID NO: 92 - protein, 262 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSLIN

SEQ ID NO: 93 - protein, 236 aa, synthetic construct
MASNFTQFVLVDNGGTGDVTVAPSNFANGVAEWISSNSRSQAYKVTCSVRQSSAQKRKYTIKVEVPKGAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQKRKYTIKVEVPKGAWRSYLNMELTIPIFATNSDCELIVKAMQGLLKDGNPIPSAIAANSLIN

SEQ ID NO: 94 - protein, 22 aa, Enterobacteria phage lambda
MDAQTRRRERRAEKQAQWKAAN

SEQ ID NO: 95 - protein, 107 aa, Enterobacteria phage lambda
MDAQTRRRERRAEKQAQWKAANPLLVGVSAKPVNRPILSLNRKPKSRVESALNPIDLTVLAEYHKQIESNLQRIERKNQRTWYSKPGERGITCSGRQKIKGKSIPLI

SEQ ID NO: 96 - RNA, 23 nt, Enterobacteria phage lambda
  source 1..23: mol_type = genomic RNA
aaacatgaggattacccatgtcg

SEQ ID NO: 97 - RNA, 23 nt, synthetic construct
  source 1..23: mol_type = other RNA
aaacatgaggatcacccatgtcg

SEQ ID NO: 98 - RNA, 15 nt, Enterobacteria phage lambda
  source 1..15: mol_type = genomic RNA
gccctgaagaagggc

SEQ ID NO: 99 - RNA, 15 nt, Enterobacteria phage lambda
  source 1..15: mol_type = genomic RNA
gccctgaaaaagggc

SEQ ID NO: 100 - DNA, 20 bp, synthetic construct
  source 1..20: mol_type = other DNA
gttcactgccgtataggcag

SEQ ID NO: 101 - RNA, 28 nt, synthetic construct
  source 1..28: mol_type = other RNA
gttcactgccgtataggcagctaagaaa

SEQ ID NO: 102 - RNA, 32 nt, synthetic construct
  source 1..32: mol_type = other RNA
nnnnnnnnnnnnnnnnnnnngttttagagcta

SEQ ID NO: 103 - RNA, 42 nt, synthetic construct
  source 1..42: mol_type = other RNA
nnnnnnnnnnnnnnnnnnnngttttagagctatgctgttttg

SEQ ID NO: 104 - RNA, 36 nt, synthetic construct
  source 1..36: mol_type = other RNA
nnnnnnnnnnnnnnnnnnnngttttagagctatgct

SEQ ID NO: 105 - RNA, 60 nt, synthetic construct
  source 1..60: mol_type = other RNA
tagcaagttaaaataaggctagtccgttatcaacttgaaaaagtggcaccgagtcggtgc

SEQ ID NO: 106 - RNA, 64 nt, synthetic construct
  source 1..64: mol_type = other RNA
agcatagcaagttaaaataaggctagtccgttatcaacttgaaaaagtggcaccgagtcggtgc

SEQ ID NO: 107 - DNA, 2279 bp, synthetic construct
  source 1..2279: mol_type = other DNA
gacgtcgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctagaaatagcaagttaaaataaggctagtccgttatcaacttgaaaaagtggcaccgagtcggtgctttttttaagcttgggccgctcgaggtacctctctacatatgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 108 - DNA, 7786 bp, synthetic construct
  source 1..7786: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacggttctcccaagaagaagaggaaagtctcgagcgactacaaagaccatgacggtgattataaagatcatgacatcgattacaaggatgacgatgacaaggctgcaggaggcggtggaagcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcc

SEQ ID NO: 109 - DNA, 7785 bp, synthetic construct
  source 1..7785: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggaggcggtggaagcgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatc

SEQ ID NO: 110 - protein, 1461 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAGGGGSGRADALDDFDLDMLGSDALDDFDLDMLGSDALDDFDLDMLGSDALDDFDLDML

SEQ ID NO: 111 - protein, 1527 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAGGGGSMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 112 - protein, 1521 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAGGGGSTAHETDKSEGGKRKADSDSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 113 - protein, 2126 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAGGGGSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV